(19) United States
(12) Patent Application Publication
(10) Pub. No.: US 2017/0136115 A1
(43) Pub. Date: May 18, 2017

(54) TRANSGENIC PLANTS EXPRESSING A TETRAVALENT CHIMERIC DENGUE VIRUS ANTIGEN TO PRODUCE EFFECTIVE VACCINES DERIVED THEREFROM

(71) Applicant: NIBIO - Norwegian Institute of Bioeconomy Research, Ås (NO)

(72) Inventor: Jihong Liu Clarke, Moss (NO)

(21) Appl. No.: 15/321,362

(22) PCT Filed: Jun. 29, 2015
(86) PCT No.: PCT/NO2015/050120

§ 371 Date: Dec. 22, 2016

(30) Foreign Application Priority Data
Jun. 27, 2014 (NO) 20140819

(51) Int. Cl. A61K 39/12; C12N 15/82; A61K 9/00; C12N 7/00

(52) U.S. Cl. CPC A61K 39/12; C12N 7/00; C12N 15/8258; A61K 9/0053; C12N 2770/24134; A61K 2039/517

(57) ABSTRACT

The present invention generally relates to the field of plant molecular biology and biotechnology as it applies to the production of plant-derived vaccines. More particularly, the present invention pertains to recombinant DNA molecules encoding a chimeric tetravalent dengue virus antigen, and vectors, transgenic plastids, transgenic plant cells, transgenic plants and transgenic parts of such plants comprising said recombinant DNA molecules as well as useful for preparing genetically transformed plant and plant cells. The invention includes plant optimized genes that encode chimeric tetravalent dengue virus antigen. The chimeric tetravalent dengue virus antigen is preferably a polyprotein comprising the immunoglobulin-like domain of the envelope protein of the dengue virus serotype-1, the immunoglobulin-like domain of the envelope protein of the dengue virus serotype-2, the immunoglobulin-like domain of the envelope protein of the dengue virus serotype-3 and the immunoglobulin-like domain of the envelope protein of the dengue virus serotype-4. The transgenic plastids, transgenic plant cells, transgenic plants and transgenic parts of such plants are preferably of the Asteraceae family.

### FIELD OF THE INVENTION

The present invention generally relates to the field of plant molecular biology and biotechnology as it applies to the production of plant-derived vaccines. More particularly, the present invention pertains to recombinant DNA molecules encoding a chimeric tetravalent dengue virus antigen, and vectors, transgenic plastids, transgenic plant cells, transgenic plants and transgenic parts of such plants comprising said recombinant DNA molecules as well as useful for preparing genetically transformed plant and plant cells. The invention includes plant optimized genes that encode chimeric tetravalent dengue virus antigen.

### BACKGROUND OF THE INVENTION

Dengue Fever is the most rapidly spreading mosquito-borne viral disease in the world threatening more than 40% of the world's population. This febrile disease is endemic in more than 100 countries in Africa, the Americas, the Eastern Mediterranean, South-east Asia and the Western Pacific. Dengue transcends international boundaries and is emerging rapidly as the consequence of globalization, rapid unplanned and unregulated urban development, improper water storage, unsatisfactory sanitary conditions, climate change and global warming. In 2010 the first European local transmission of dengue was reported in France and Croatia and in 2012 an outbreak of dengue on Madeira islands of Portugal resulted in over 2 000 cases (WHO, 2013). Infection with the Dengue viruses can cause dengue fever (DF), dengue haemorrhagic fever (DHF) and dengue shock syndrome (DSS). Dengue fever is a flu-like illness accompanied by symptoms like headache, pain behind the eyes, muscle and joint pains, nausea, vomiting, swollen glands or rash. The severe forms DHF and DSS are potentially deadly complications due to plasma leaking, fluid accumulation, respiratory distress, severe bleeding or organ impairment (WHO, 2013). Dengue infections are a significant cause of morbidity and mortality and lead to adverse social and economic impacts in many developing tropical countries. WHO estimates 50-100 million new infections occurring each year (WHO, 2013) and an additional 500 000 cases of DHF/DSS and over 20 000 dengue related deaths each year (WHO, 2006).

Cases reported to the World Health Organization (WHO) over the past four decades show an upward trend, partly resulting from an increased spread of vector mosquitoes and increase in total human population, including specific increases in urban populations at risk of dengue infection. Dengue is endemic in the South East Asian region and is a leading cause of hospitalization and death among children in several countries in the region (WHO, 2010). Dengue affects all strata of the population, but flourishes in urban slums and poor peri-urban areas. Dengue is a matter of much concern in India, especially to low income groups, who do not have timely access to medical and diagnostical facilities and due to inadequate mosquito control.

The Dengue virus belongs to the genus Flavivirus, family Flaviviridae (Calisher et al, 1989) and its genome is a ˜11 kb long positive single stranded RNA. The RNA is transcribed as polycistrons and the polyprotein undergoes post-translational cleavage by viral and host proteases generating three structural and 7 non-structural proteins. The enveloped virus particles are of icosahedral shape with a diameter of 500 Å corresponding to 50 nm. The virus is typically transmitted by the bite of the blood feeding, day active mosquito Aedes aegypti (WHO, 2009) and until recently was believed to occur in four closely related but antigenically and genetically distinct serotypes DEN-1, DEN-2, DEN-3 and DEN-4.

Infection with any one serotype usually causes the mild form of the disease (dengue fever) and provides lifelong homologous immunity to that serotype with only transient cross protection against the others. However, subsequent infection with a different serotype leads to the life-threatening forms of the disease: DHF and DSS. Dengue pathogenesis appears to be the result a complex interaction of host and viral factors, with the two most evident contributors being antibody dependent enhancement and inherent virulence of the dengue viruses.

The most effective way to reduce disease and mortality rate from infectious diseases is to vaccinate susceptible populations in risk. The development of a vaccine offers the potential for effective prevention and long-term control of virus infection. Despite this, more than 60 years after the discovery of the virus and the start of systematic research into dengue vaccines, no such vaccine has been brought to the market so far (WHO, 2013). The development of the vaccine has been hindered and delayed by the complex pathology of the disease, by the need to control four viruses simultaneously and by the lack of a suitable animal model. However the increasing spread and intensity of the disease over the past years has triggered new interest and investment in dengue vaccine research.

Infection with any one of the dengue serotypes provides lifelong immunity to that serotype, but secondary infection with a different serotype can predispose an individual to potentially fatal DHF and DSS. This is because anti-dengue antibodies specific to one serotype do cross-react with the remaining serotypes, but do not cross-protect against them (Halstead, 1988). This has prompted the view that an efficient dengue vaccine must be tetravalent, procuring protection against all four virus serotypes at once (Hombach et al, 2005). The main strategies applied for vaccine production against dengue fever consist in traditionally and molecularly attenuated live viruses, chimeric live virus vaccines, vector based vaccines, DNA vaccines and recombinant subunit vaccines (Guzman et al, 2009).

The most advanced dengue vaccine candidates have been developed as single serotype-specific vaccine formulations (monovalent vaccines) and are being evaluated as physical four-in-one mixtures for their capacity to elicit protective immunity against the four serotypes (Swaminathan et al, 2010). Empirically attenuated vaccine strains for all four dengue serotypes have been obtained by repeated serial passage in primary dog kidney cells (Halstead & Marchette, 2003) by two independent research groups. The Mahidol University in Thailand (Bhamarapravati & Yoksan, 2001) licensed their vaccine candidate strains to Sanofi Pasteur (France, Mahidol vaccine), while the Walter Reed Army Institute of Research (Eckels et al, 2000b) licensed their candidate strains to GlaxoSmithKline (Belgium, WRAIR vaccine) for large scale production and further evaluation. Repeated reports of unbalanced immune responses in human trials using the attenuated tetravalent vaccine formulations of both, the Mahidol (Edelman et al, 2003; Kanesa-thasan et al, 2001) and the WRAIR vaccine (Eckels et al, 2000a; Edelman et al, 2003; Sun et al, 2003) have stalled further development and commercialization of these vaccine candidates.

An alternate strategy has been adopted by Sanofi-Pasteur where the structural genes of the empirically attenuated Yellow fever virus strain 17D (YF17D, (Monath, 1997) were replaced with the premembrane (prM) and envelop (E) gene of the dengue viruses to create four monovalent chimeric yellow fever dengue vaccine strains (CYD strains, (Guirakhoo et al, 2001). Immunization of monkeys with a tetravalent vaccine formulation resulted again in an unbalanced immune response with the highest response being directed against DEN-2 (Guirakhoo et al, 2001). However, after several dose adjustments and promising results of an administration study in healthy adult volunteers (Morrison et al, 2010), the tetravalent CYD formulation entered Phase II trials. The results reported by the pediatric phase 2b trial in Thailand showed good protection against DEN-1, DEN-3 and DEN-4, but not against DEN-2 (Sabchareon et al, 2012). Currently, the tetravalent vaccine candidate CYD15 is undergoing a phase III clinical trial in dengue-endemic areas in Latin America with the scope of evaluating the efficacy and safety of protection in healthy children and adolescents aged 9 to 16 years (Pasteur, 2011).

Other approaches include a deletion of 30 nucleotides in the viral 3′UTR (Δ30 vaccines, (Durbin et al, 2001), intertypic chimeric dengue vaccines (Bhamarapravati et al, 1996) self-destructing virus mutants with a furin protease cleavage site in the membrane glycoprotein (Brown, 2004), RepliVax vectors that undergo only one cycle of infection in the vaccinated host (Frolov et al, 2007) and the utilization of the live attenuated Schwarz measles virus vaccine as a carrier for dengue antigenes (Brandler et al, 2007).

Although the current tetravalent dengue vaccine candidates, which are in advanced stages of development are based on live-attenuated virus strains or genetically manipulated chimeric Flavivirus, the continuous difficulties associated with these vaccine candidates have necessitated the exploration of alternative non-replicating subunit vaccines. The majority of attempts to produce a recombinant protein based vaccine focus on the envelope (E) protein of dengue viruses.

The E protein consists of three domains (Modis et al, 2003): the envelop domain I (EDI), flanked by a dimerization domain (EDII) containing the fusion peptide and an immunoglobulin-like domain (EDIII) which contains the host cell surface receptor binding motif (Chen et al, 1997) and several serotype specific neutralizing epitopes (Chin et al, 2007; Megret et al, 1992). The EDIII protrudes from the virus surface to facilitate binding to the host cell surface receptor (Crill & Roehrig, 2001) and mediates host membrane fusion (Allison et al, 2001). This EDIII domain spanning amino acids 300-400 of the E protein has emerged as the most promising region for vaccine development (Guzman et al, 2010) since it appears to have only very low intrinsic potential for eliciting cross-reactive antibodies against heterologous serotypes (Hombach et al, 2005).

Recombinant EDIII antigens have been produced using bacteria—(e.g., McDonald et al, 2009), yeast—(Ivy et al, 2000) and plant expression systems (e.g., Martinez et al, 2010). In order to avoid the unbalanced immune response elicited by tetravalent formulations consisting of stoichiometrically mixed monovalent vaccines, a recombinant fusion protein linking the EDIII domain of dengue virus serotypes 1, 2, 3 and 4 has been developed. This construct was reported to elicit neutralizing antibodies against all four serotypes (Batra et al, 2007; Etemad et al, 2008).

WHO and other organizations have stressed the need for new technologies to create vaccines where the cost of delivery can be decreased, making the vaccines more affordable and more accessible to populations in developing and poor economies.

Accordingly, it is an object of the present invention to provide vaccines against dengue virus that are safe and cost effective and that will be affordable to end users in developing countries.

### SUMMARY OF THE INVENTION

The present invention provides in a first aspect recombinant DNA molecules comprising a nucleotide sequence that, when expressed in a plant cell, encodes a tetravalent dengue (DEN) virus antigen. Particularly, the present invention provides a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The present invention provides in a further aspect vectors as detailed herein. Particularly, the present invention provides a vector, such as a transformation vector, comprising a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The present invention provides in a further aspect transgenic plastids as detailed herein. Particularly, the present invention provides a plastid, such as a chloroplast, comprising a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The present invention provides in a further aspect transgenic plant cells as detailed herein. Particularly, the present invention provides a transgenic plant cell, such as a transgenic Lactuca sativa cell, comprising a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The present invention provides in a further aspect transgenic plant cells comprising a plastid as detailed herein.

The present invention provides in a further aspect transgenic plants or transgenic parts of said plants as detailed herein. Particularly, the present invention provides a transgenic plant or transgenic part of said plant, such as a transgenic Lactuca sativa plant, comprising a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The present invention provides in a further aspect transgenic plants or transgenic parts of said plants comprising a plurality of plant cells as detailed herein.

The present invention provides in a further aspect transgenic seeds as detailed herein. Particularly, the present invention provides a transgenic seed, such as transgenic seed derived from a Lactuca sativa plant, comprising a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The present invention provides in a further aspect methods of producing a transgenic plastid as detailed herein. Particularly, the present invention provides a method for producing a transgenic plastid comprising the step of introducing a recombinant DNA molecule or vector as detailed herein into a plastid, such as a chloroplast.

The present invention provides in a further aspect methods of producing a transgenic plant cell, a transgenic plant or a transgenic plant part as detailed herein. Particularly, the present invention provides a method of producing a transgenic plant cell, a transgenic plant or a transgenic plant part comprising the step of introducing a recombinant DNA molecule or a vector as detailed herein into a plant cell, a plant or plant part, such as Lactuca sativa cells or Lactuca sativa plant.

The present invention provides in a further aspect methods of producing a transgenic plant using a transgenic seed as detailed herein. Particularly, the present invention provides a method of producing a transgenic plant comprising the steps of (a) planting a transgenic seed as detailed herein; and (b) growing a plant from said seed.

The present invention provides in a further aspect methods of producing a tetravalent chimeric dengue virus antigen. Particularly, the present invention provides a method of producing a tetravalent chimeric dengue virus antigen comprising the steps of a) producing a transgenic plant as detailed herein, and b) incubating said plant under conditions wherein said plant expresses said antigen.

The present invention provides in a further aspect transgenic plant cells, transgenic plants or transgenic parts of said plants as detailed herein for use in vaccination. Particularly, the present invention provides a transgenic plant cell, transgenic plant or transgenic part of said plant as detailed herein for use in vaccinating a subject, such as a human, against dengue virus.

The present invention provides in a further aspect vaccines as detailed herein. Particularly, the present invention provides a vaccine comprising a transgenic plant cell, a transgenic plant or a transgenic part of said plant as detailed herein optionally together with one or more pharmaceutically acceptable excipients.

The present invention provides in a further aspect methods of producing a vaccine. Particularly, the present invention provides a method of producing a vaccine comprising the steps of a) Producing a transgenic plant cell, a transgenic plant or a transgenic part of said plant comprising a recombinant DNA molecule as detailed herein, b) incubating said transgenic plant cell, said transgenic plant or said transgenic part of said plant under conditions wherein said plant cell, plant or part of said plant expresses the polyprotein encoded by said recombinant DNA molecule, c) harvesting said transgenic plant cell, said transgenic plant or said transgenic part of said plant, and d) formulating said transgenic plant cell, said transgenic plant or said transgenic part of said plant into a vaccine, optionally together with one or more pharmaceutically acceptable excipients.

The present invention provides in a further aspect vaccination methods. Particularly, the present invention provides a method of vaccinating a subject, such as a human, against dengue virus, the method comprising the step of administering an effective amount of a vaccine as detailed herein to a subject.

The present invention provides in a further aspect the use of a transgenic plant cell, a transgenic plant or a transgenic part of said plant as detailed herein in the manufacture of a medicinal product. Particularly, the present invention provides the use of a a transgenic plant cell, a transgenic plant or a transgenic part of said plant as detailed herein in the manufacture of a medicinal product for vaccination of a subject, such as a human, against dengue virus.

The present invention provides in a further aspect isolated nucleic acid molecule encoding the polyprotein as detailed herein. Particularly, the present invention provides an isolated nucleic acid molecule comprising the nucleotide sequences set forth in SEQ ID NOS: 7, 8, 9 and 10 or nucleotide sequences at least about 80%, such as at least about 85%, at least about 90% at least about 93%, at least about 95%, at least about 96%, at least about 97%, at least about 98% or at least about 99%, identical thereto.

### BRIEF DESCRIPTION OF THE DRAWINGS

FIG. 1: Schematic representation of the expression vactors for the generation of transplastomic lettuce plants. a) The final lettuce specific plastid transformation vector pEXP-PN-goi-L; b) Wild-type lettuce plastid genome (CP); c) lettuce plastid genome with integrated transgene expression cassette for EDIII 1 and EDIII 1-4, respectively. The Southern Blot probe (trnA) is shown as an arrow and the expected SmaI fragments are shown as arrows and their size is indicated next to the respective goi. aadA: spectinomycin resistance gene; Amp(R): ampicillin resistance gene; attB1/attB2: Gateway® recombination sites; INSL*/INSR*: lettuce specific left/right insertion site; trnI/trnA: sequences coding for tRNA-I/tRNA-A; EDIII 1/1-4: coding sequence for transgene including a Hexa-his-tag; PsbA: tobacco psbA promoter (Staub & Maliga, 1993); Prrn16: tobacco rrn16 PEP+NEP promoter (Ye et al, 2001); 3′(C): 3′UTR of Chlamydomonas rbcL gene; 5′psbA: 5′UTR of tobacco psbA gene; 3′(T): 3′UTR of tobacco rbcL gene; ORI: bacterial origin of replication. p296/p297: primers used for PCR and the corresponding PCR product is shown as a dotted line marked with its molecular weight for both transgenes.

FIG. 2: PCR and Southern blot analysis of lettuce transformants expressing the chimeric tetravalent EDIII antigen. a) PCR using primer p296/p297 binding inside the transgene expression cassette. The expected PCR product is 1841 bp for S12-PN-EDIII 1-4 (lane 1) or 836 bp for S16-PN-EDIII 1 (lane 2). b) Southern blot analysis of DNA isolated from regenerated plant lines and wild-type (wt) was performed using a 665 bp DIG labeled probe that binds inside the trnA region (INSR) of the plastid genome. The expected fragment size after SmaI digest is 6533 bp (for S12-PN-EDIII 1-4) or 5545 bp (for S16-PN-EDIII 1) and 3130 bp for wt plants. The positions of primers and the corresponding expected size of PCR product, as well as restriction sites, probe position and the size of expected Southern blot bands are indicated in FIG. 1. M: 1 kb DNA ladder, NEB.

FIG. 3: Phenotype and Germination assay of transplastomic lettuce plants. a) plants growing in the greenhouse; b) flowering plants; c) one week old seedlings obtained from transplastomic plants and wild-type seeds germinated on spectinomycin (30 mg/L) containing medium.

FIG. 4: Western Blot analysis. a) TP and TSP isolated from plant line S12-PN-EDIII 1-4; b) TP and TSP isolated from plant line S16-PN-EDIII 1; the amount of TSP/TP loaded is given above the respective lane in μg; in a) 20 μg TP/TSP were loaded for the wild-type, while in b) 50 μg TP/TSP were loaded for the wild-type. The arrows indicate the 47 kDa EDIII 1-4 and the 13 kDa EDIII 1; M: spectra multicolour broad range protein ladder (Thermo Scientific), molecular size indicated in kDa.

### DETAILED DESCRIPTION OF THE INVENTION

Unless specifically defined herein, all technical and scientific terms used have the same meaning as commonly understood by a skilled artisan in the fields of biochemistry, genetics, molecular biology and immunology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Recombinant DNA Molecules and Vectors

As indicated above, the present invention recombinant DNA molecules comprising a nucleotide sequence that, when expressed in a plant cell, encodes a tetravalent dengue (DEN) virus antigen.

Particularly, the present invention provides a recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

The immunoglobulin-like domain of the envelope (E) protein of the dengue (DEN) virus serotype 1 (EDIII-1) may comprise the amino acid sequence set forth in SEQ ID NO: 1. A variant of EDIII-1 may comprise an amino acid sequence which has at least about 70%, such as at least about 75%, at least about 80%, at least about 85%, at least about 90%, at least about 93%, at least about 95%, at least about 96%, at least about 97%, at least about 98% or at least about 99%, sequence identify to the amino acid sequence set forth in SEQ ID NO: 1. According to certain embodiments, a variant of EDIII-1 may comprise an amino acid sequence which has at least about 90%, such as at least about 93%, at least about 95%, at least about 96%, at least about 97%, at least about 98% or at least about 99%, sequence identify to the amino acid sequence set forth in SEQ ID NO: 1. According to certain other embodiments, a variant of EDIII-1 may comprise an amino acid sequence which has at least about 95%, such as at least about 96%, at least about 97%, at least about 98% or at least about 99%, sequence identify to the amino acid sequence set forth in SEQ ID NO: 1. Preferably, the variant is an immunogenic variant which means that it capable of eliciting an immune response upon administration to a subject.

The immunoglobulin-like domain of the envelope (E) protein of the dengue (DEN) virus serotype 2 (EDIII-2) may comprise the amino acid sequence set forth in SEQ ID NO: 2. A variant of EDIII-2 may comprise an amino acid sequence which has at least about 70%, such as at least about 75%, at least about 80%, at least about 85%, at least about 90%, at least about 93%, at least about 95% at least about 96%, at least about 97%, at least about 98% or at least about 99%, sequence identify to the amino acid sequence set forth in SEQ ID NO: 2. According to certain embodiments, a variant of EDIII-2 may comprise an amino acid sequence which has at least about 90%, such as at least about 93%, at least about 95%, at least about 96%, at least about 97%, at least about 98% or at least about 99%, sequence identify to the amino acid sequence set forth in SEQ ID NO: 2. According to certain other embodiments, a variant of EDIII-2 may comprise an amino acid sequence which has at least about 95%, such as at least about 96%, at least about 97%, at least about 98% or at least about 99%, sequence identify to the amino acid sequence set forth in SEQ ID NO: 2. Preferably, the variant is an immunogenic variant which means that it capable of eliciting an immune response upon administration to a subject.

The immunoglobulin-like domain of the envelope (E) protein of the dengue (DEN) virus serotype 3 (EDIII-3) may comprise the amino acid sequence set forth in SEQ ID NO: 3. A variant of EDIII-3 may comprise an amino acid sequence which has at least about 70%, such as at least about 75%, at least about 80%, at least about 85%, at least about 90%, at least about 93%, at least about 95% at least about 96%, at least about 97%, at least about 98% or at least about 99%, sequence identify to the amino acid sequence set forth in SEQ ID NO: 3. According to certain embodiments, a variant of EDIII-3 may comprise an amino acid sequence which has at least about 90%, such as at least about 93%, at least about 95%, at least about 96%, at least about 97%, at least about 98% or at least about 99%, sequence identify to the amino acid sequence set forth in SEQ ID NO: 3. According to certain other embodiments, a variant of EDIII-3 may comprise an amino acid sequence which has at least about 95%, such as at least about 96%, at least about 97%, at least about 98% or at least about 99%, sequence identify to the amino acid sequence set forth in SEQ ID NO: 3. Preferably, the variant is an immunogenic variant which means that it capable of eliciting an immune response upon administration to a subject.

The immunoglobulin-like domain of the envelope (E) protein of the dengue (DEN) virus serotype 4 (EDIII-4) may comprise the amino acid sequence set forth in SEQ ID NO: 4. A variant of EDIII-4 may comprise an amino acid sequence which has at least about 70%, such as at least about 75%, at least about 80%, at least about 85%, at least about 90%, at least about 93%, at least about 95% at least about 96%, at least about 97%, at least about 98% or at least about 99%, sequence identify to the amino acid sequence set forth in SEQ ID NO: 4. According to certain embodiments, a variant of EDIII-4 may comprise an amino acid sequence which has at least about 90%, such as at least about 93%, at least about 95%, at least about 96%, at least about 97%, at least about 98% or at least about 99%, sequence identify to the amino acid sequence set forth in SEQ ID NO: 4. According to certain other embodiments, a variant of EDIII-4 may comprise an amino acid sequence which has at least about 95%, such as at least about 96%, at least about 97%, at least about 98% or at least about 99%, sequence identify to the amino acid sequence set forth in SEQ ID NO: 4. Preferably, the variant is an immunogenic variant which means that it capable of eliciting an immune response upon administration to a subject.

The four EDIIIs or variants thereof may be arranged in the polyprotein in any possible order.

The polyprotein may comprise, arranged in the N-terminal to C-terminal direction, EDIII-1, EDIII-2, EDIII-3 and EDIII-4. The polyprotein may comprise, arranged in the N-terminal to C-terminal direction, EDIII-1, EDIII-3, EDIII-4 and EDIII-2. The polyprotein may comprise, arranged in the N-terminal to C-terminal direction, EDIII-1, EDIII-4, EDIII-2 and EDIII-3. The polyprotein may comprise, arranged in the N-terminal to C-terminal direction, EDIII-1, EDIII-2, EDIII-4 and EDIII-3. The polyprotein may comprise, arranged in the N-terminal to C-terminal direction, EDIII-1, EDIII-3, EDIII-2 and EDIII-4. The polyprotein may comprise, arranged in the N-terminal to C-terminal direction, EDIII-1, EDIII-4, EDIII-3 and EDIII-2.

The polyprotein may comprise, arranged in the N-terminal to C-terminal direction, EDIII-2, EDIII-1, EDIII-3 and EDIII-4. The polyprotein may comprise, arranged in the N-terminal to C-terminal direction, EDIII-2, EDIII-1, EDIII-4 and EDIII-3. The polyprotein may comprise, arranged in the N-terminal to C-terminal direction, EDIII-2, EDIII-3, EDIII-1 and EDIII-4. The polyprotein may comprise, arranged in the N-terminal to C-terminal direction, EDIII-2, EDIII-3, EDIII-4 and EDIII-1. The polyprotein may comprise, arranged in the N-terminal to C-terminal direction, EDIII-2, EDIII-4, EDIII-3 and EDIII-1. The polyprotein may comprise, arranged in the N-terminal to C-terminal direction, EDIII-1, EDIII-4, EDIII-2 and EDIII-1.

The polyprotein may comprise, arranged in the N-terminal to C-terminal direction, EDIII-3, EDIII-2, EDIII-1 and EDIII-4. The polyprotein may comprise, arranged in the N-terminal to C-terminal direction, EDIII-3, EDIII-2, EDIII-4 and EDIII-1. The polyprotein may comprise, arranged in the N-terminal to C-terminal direction, EDIII-3, EDIII-1, EDIII-2 and EDIII-4. The polyprotein may comprise, arranged in the N-terminal to C-terminal direction, EDIII-3, EDIII-1, EDIII-4 and EDIII-2. The polyprotein may comprise, arranged in the N-terminal to C-terminal direction, EDIII-3, EDIII-4, EDIII-1 and EDIII-2. The polyprotein may comprise, arranged in the N-terminal to C-terminal direction, EDIII-3, EDIII-4, EDIII-2 and EDIII-1.

The polyprotein may comprise, arranged in the N-terminal to C-terminal direction, EDIII-4, EDIII-1, EDIII-2 and EDIII-3. The polyprotein may comprise, arranged in the N-terminal to C-terminal direction, EDIII-4, EDIII-1, EDIII-3 and EDIII-2. The polyprotein may comprise, arranged in the N-terminal to C-terminal direction, EDIII-4, EDIII-2, EDIII-3 and EDIII-1. The polyprotein may comprise, arranged in the N-terminal to C-terminal direction, EDIII-4, EDIII-2, EDIII-1 and EDIII-3. The polyprotein may comprise, arranged in the N-terminal to C-terminal direction, EDIII-4, EDIII-3, EDIII-1 and EDIII-2. The polyprotein may comprise, arranged in the N-terminal to C-terminal direction, EDIII-4, EDIII-3, EDIII-2 and EDIII-1.

It is understood that each of the four EDIIIs may be replaced by its respective variant. Thus, the foregoing arrangement in the polyprotein applies equally to variants and compositions of EDIIIs and variants.

The EDIIIs (or variants thereof) may either be directly linked to each other or may be joined by a linker. Therefore, according to certain embodiments, the EDIIIs (or variants thereof) are directly linked to each other. According to other certain embodiments, the adjacent EDIIIs are joined by linkers. The linkers may each independently be a peptide linker, such as polyglycine linker. Peptide linkers are generally composed of one or more amino acids which may be naturally and/or non-naturally occurring amino acids, such as glycine.

A peptide linker employed in accordance with the invention may be composed of at least about one amino acid, such as at least about 2 amino acids, at least about 3 amino acids, at least about 4 amino acids, at least about 5 amino acids, at least about 6 amino acids, at least about 7 amino acids, at least about 8 amino acids, at least about 9 amino acids, at least about 10 amino acids, or at least about 15 amino acids. Hence, a peptide linker may be composed of at least 5 amino acids. For example, a peptide linker employed in accordance with the invention may be a pentaglycine linker.

A peptide linker employed in accordance with the invention may be composed of from about 1 to about 20 amino acids, such as from about 2 to about 15 amino acids, from about 2 to about 10, from about 2 to about 7, from about 2 to about 6, from about 2 to about 5, from about 3 to about 15, from about 3 to about 10, from about 3 to about 7, from about 3 to about 6, from about 3 to about 5, from about 4 to about 15, from about 4 to about 10, from about 4 to about 7, from 4 to about 6, from about 4 to about 5, from about 5 to about 15, from about 5 to about 10, from about 5 to about 7, or from about 5 to about 6, such as about 5 amino acids.

According to certain embodiments, the recombinant DNA molecule comprises a nucleotide sequence encoding a polyprotein which comprises the amino acid sequence set forth in SEQ ID NO: 5 or an amino acid sequence having at least about 80%, such as at least about 85%, at least about 90%, at least about 93%, at least about 95%, at least about 96%, at least about 97%, at least about 98% or at least about 99%, sequence identify to the amino acid sequence set forth in SEQ ID NO: 5. According to particular embodiments, the recombinant DNA molecule comprises a nucleotide sequence encoding a polyprotein which comprises the amino acid sequence set forth in SEQ ID NO: 5 or an amino acid sequence having at least about 90%, such as at least about 93%, at least about 95%, at least about 96%, at least about 97%, at least about 98% or at least about 99%, sequence identify to the amino acid sequence set forth in SEQ ID NO: 5. According to other particular embodiments, the recombinant DNA molecule comprises a nucleotide sequence encoding a polyprotein which comprises the amino acid sequence set forth in SEQ ID NO: 5 or an amino acid sequence having at least about 95%, such as at least about 96%, at least about 97%, at least about 98% or at least about 99%, sequence identify to the amino acid sequence set forth in SEQ ID NO: 5. Preferably, such variant is capable of eliciting an immune response upon administration to a subject.

According to certain embodiments, the recombinant DNA molecule comprises the nucleotide sequence set forth in SEQ ID NO: 6 or a nucleotide sequence having at least about 70%, such as at least about 75%, at least about 80%, at least about 85%, at least about 90%, at least about 93%, at least about 95%, at least about 96%, at least about 97%, at least about 98% or at least about 99%, sequence identify to the nucleotide sequence set forth in SEQ ID NO: 6. According to particular embodiments, the recombinant DNA molecule comprises the nucleotide sequence set forth in SEQ ID NO: 6 or a nucleotide sequence having at least about 90%, such as at least about 93%, at least about 95%, at least about 96%, at least about 97%, at least about 98% or at least about 99%, sequence identify to the nucleotide sequence set forth in SEQ ID NO: 6. According to other particular embodiments, the recombinant DNA molecule comprises the nucleotide sequence set forth in SEQ ID NO: 6 or a nucleotide sequence having at least about 95%, such as at least about 96%, at least about 97%, at least about 98% or at least about 99%, sequence identify to the nucleotide sequence set forth in SEQ ID NO: 6.

According to certain embodiments, the recombinant DNA molecule comprises the nucleotide sequences set forth in SEQ ID NOS: 7, 8, 9 and 10, or nucleotide sequences having at least about 70%, such as at least about 75%, at least about 80%, at least about 85%, at least about 90%, at least about 93%, at least about 95%, at least about 96%, at least about 97%, at least about 98% or at least about 99%, sequence identity to the nucleotide sequences set forth in SEQ ID NOS: 7, 8, 9, and 10, respectively. According to particular embodiments, the recombinant DNA molecule comprises the nucleotide sequences set forth in SEQ ID NOS: 7, 8, 9 and 10, or nucleotide sequences having at least about 90%, such as at least about 93%, at least about 95%, at least about 96%, at least about 97%, at least about 98% or at least about 99%, sequence identity to the nucleotide sequences set forth in SEQ ID NOS: 7, 8, 9, and 10, respectively. According to other particular embodiments, the recombinant DNA molecule comprises the nucleotide sequences set forth in SEQ ID NOS: 7, 8, 9 and 10, or nucleotide sequences having at least about 95%, such as at least about 96%, at least about 97%, at least about 98% or at least about 99%, sequence identity to the nucleotide sequences set forth in SEQ ID NOS: 7, 8, 9, and 10, respectively.

Promoters useful in accordance with the invention are any known promoters that are functional in a plant cell to cause the production of an mRNA molecule. Many such promoters are known to the skilled person. Such promoters include promoters normally associated with other genes, and/or promoters isolated from any plant cell. The use of promoters for protein expression is generally known to those of skilled in the art of molecular biology, for example, see Sambrook et al., Molecular cloning: A Laboratory Manual, Cold Spring Harbor Laboratory, Cold Spring Harbor, N.Y., 1989. The promoter employed may be inducible. The term “inducible” used in the context of a promoter means that the promoter only directs transcription of an operably linked nucleotide sequence if a stimulus is present, such as a change in temperature or the presence of a chemical substance (“chemical inducer”). As used herein, “chemical induction” according to the present invention refers to the physical application of a exogenous or endogenous substance (incl. macromolecules, e.g., proteins or nucleic acids) to a plant cell or plant (e.g., by spraying a liquid solution comprising a chemical inducer thereon, such as on the leaves of a plant, application to the roots or exposing the plant cell or plant to gas or vapour). This has the effect of causing the target promoter present in the plant cell or cells of the plant to increase the rate of transcription. Alternatively, the promoter employed may be constitutive. The term “constitutive” used in the context of a promoter means that the promoter is capable of directing transcription of an operably linked nucleotide sequence in the absence of stimulus (such as heat shock, chemicals etc.).

Non-limiting examples of plant functional promoters are the Lactuca sative psbA promoter, the tabacco psbA promoter, the tobacco rrn16 PEP+NEP promoter, the CaMV 35S promoter, the 19S promoter, the tomate E8 promoter, the nos promoter, the Mac promoter, the pet E promoter or the ACT1 promoter.

Preferable, the promoter employed in accordance with the present invention is a promoter functional in a plant cell of the Asteraceae family, particularly in a plant cell of the Lactuca genus, and more particularly in a Lactuca sativa cell.

According to certain embodiments, the promoter is the Lactuca sative psbA promoter or tabacco psbA promoter. According to particular embodiments, the promoter is the Lactuca sative psbA promoter. According to other particular embodiments, the promoter is the tabacco psbA promoter.

The recombinant DNA molecule may further comprising at least one regulatory element selected from the group consisting of a 5′ untranslated region (5′ UTR), 3′ untranslated region (3′ UTR), and transit peptide region.

In accordance with certain embodiments, the recombinant DNA molecule comprises a 5′ untranslated region. Many 5′ UTRs are known to the skilled person. Such 5′ UTRs include 5′ UTR normally associated with other genes, in particular genes found in plant cells. Non-limiting examples of 5′ UTRs suitable in accordance with the present invention are the 5′ UTR of the Lactuca sative psbA gene and the 5′ UTR of the tabacco psbA gene.

In accordance with certain embodiments, the recombinant DNA molecule comprises a 3′ untranslated region. The 3′ untranslated region functions in said plant cell to cause the termination of transcription and the addition of polyadenolyted ribonucleotides to the 3′ end of an mRNA molecule. Many 3′ UTRs are known to the skilled person. Such 3′ UTRs include 3′ UTR normally associated with genes, in particular genes found in plant cells. Non-limiting examples of 3′ UTRs suitable in accordance with the present invention are the 3′ UTR of the Lactuca sative psbA gene, the tabacco psbA gene and the tabacco rbcL gene.

The recombinant DNA molecule according to the present invention may be part of a vector. Accordingly, the present invention also provides a vector, such as a transformation vector, comprising a recombinant DNA molecule as detailed herein. The vector may be an expression vector or a transformation vector.

According to certain embodiments, the vector is a transformation vector, and more particularly a plastid transformation vector for stably transforming a plastid. For stable transformation of a plastid, such as a chloroplast, the vector may comprise a first flanking DNA sequence and second flanking DNA sequence each of which is homologous to sequences in a spacer region of the genome of said plastid. The first and second flanking DNA sequences are positioned 5′ and 3′ to the sequence of the recombinant DNA molecule, respectively. The spacer region of the plastid may for instance be a transcriptionally active spacer region, such as the transcriptionally active spacer region between the trnI and trnA genes in the plastid genome. The flanking DNA sequences thus allow the site-directed integration of the recombinant DNA molecule into the spacer region of the plastid genome via homologous recombination.

According to certain embodiments, the vector comprises a first flanking DNA sequence and second flanking DNA sequence which are homologous the trnI coding sequence and the trnA coding sequence, respectively.

The vector may thus comprise, as operably linked components arranged in the 5′ to 3′ direction, said first flanking DNA sequence, said promoter, said 5′ untranslated region, said nucleotide sequence encoding the polyprotein, said 3′ untranslated region, and said second flanking DNA sequence.

### Transgenic Plastids, Plant Cells and Plants

The present invention provides transgenic plastids, plant cells and plants expressing the polyprotein as detailed herein.

The present invention provides transgenic plastids. Particularly, a transgenic plastid according to the invention is a plastid, such as a chloroplast, comprising a recombinant DNA molecule as detailed herein. Preferably, said transgenic plastid expresses the polyprotein encoded by the recombinant DNA molecule.

According to certain embodiments, the recombinant DNA molecule is stably intergrated into the genome of the transgenic plastid of the invention.

The plastid may be a chloroplast, chromoplast, gerentoplast, etioplast or leucoplast (such as an amyloplast, proteinoplast or elaioplast). According to certain embodiments, the plastid is a chloroplast. The plastid may be derived from any known plant cell. According to certain embodiments, the plastid is derived from a plant or plant cell of the Asteraceae family, particularly from a plant or plant cell of the Lactuca genus, and more particularly from a Lactuca sativa plant or cell.

The present invention also provides transgenic plant cells. Particularly, a transgenic plant cell, such as a transgenic Lactuca sativa cell, according to the present invention comprises a recombinant DNA molecule as detailed herein. Preferably, said transgenic plant cell expresses the polyprotein encoded by the recombinant DNA molecule.

The recombinant DNA molecule may be stably integrated in a genome of the plant cell. Particularly, the recombinant DNA molecule may be stably integrated into the genome of a plant cell plastid or may be stably integrated into a chromosome of the plant cell's nucleus. The transgenic plant cell may also be a plant cell comprising a transgenic plastid as detailed herein.

The transgenic plant cell may be (or derived from) any know plant cell. According to certain embodiments, the transgenic plant cell is a plant cell of the Asteraceae family, particularly a plant cell of the Lactuca genus, and more particularly a Lactuca sativa plant cell.

The transgenic plant cell may be a plant cell in a grown plant or a plant seed.

The present invention also provides transgenic plants or transgenic parts of said plants. Particularly, a transgenic plant or transgenic part of said plant, such as a transgenic Lactuca sativa plant or part thereof, according to the present invention comprises a recombinant DNA molecule as detailed herein. Preferably, said transgenic plant expresses the polyprotein encoded by the recombinant DNA molecule.

A transgenic part of the transgenic plant of the invention may be any part of said plant, such as a leaf or root. With “transgenic part” of a plant it is meant that said part, such as leaf(s), and more particularly cells thereof, comprises a recombinant DNA molecule as detailed herein. Preferably, said transgenic part expresses the polyprotein encoded by the recombinant DNA molecule.

The recombinant DNA molecule may be stably integrated in a genome of a plant cell. Particularly, the recombinant DNA molecule may be stably integrated into the genome of a plant cell plastid or may be stably integrated into a chromosome of a plant cell's nucleus.

The transgenic plant or transgenic part of said plant may comprise a plurality of transgenic plant cells as detailed herein.

The transgenic plant may be (or derived from) any know plant. According to certain embodiments, the transgenic plant is a plant of the Asteraceae family, particularly a plant of the Lactuca genus, and more particularly a Lactuca sativa plant.

The present invention also provides transgenic seeds. Particularly, a transgenic seed, such as transgenic seed derived from a Lactuca sativa plant, comprises a recombinant DNA molecule as detailed herein. A transgenic seed according to the present invention may comprise a plurality of transgenic plant cells as detailed herein.

The present invention also provides methods of producing transgenic plastids, transgenic plant cells and transgenic plants or transgenic parts of such plants. In general, such methods comprise the step of introducing a recombinant DNA molecule or vector as detailed herein into a plastid, such as a chloroplast, a plant cell or a plant.

The plant or plant cell that can be used for practice of the present invention include any dicotyledon and monocotyledon. These include, but are not limited to, tobacco, tomato, potato, eggplant, pepino, yam, pea, sugar beet, lettuce, bell pepper, celery, carrot, asparagus, onion, grapevine, muskmelon, strawberry, rice, sunflower, wheat, oats, maize, cotton, walnut, spruce/conifer, poplar and apple, berries such as strawberries, raspberries, alfalfa and banana. Since many edible plants used by humans for food or as components of animal feed are dicotyledenous plants, dicotyledons are typically employed. The invention includes whole plants, plant cells, plant organs, plant tissues, plant seeds, protoplasts, callus, cell cultures, and any group of plant cells organized into structural and/or functional units capable of expressing a polyprotein of the present invention.

According to certain embodiments, the plant or plant cell is a plant or plant cell of the Asteraceae family, particularly a plant or plant cell of the Lactuca genus, and more particularly a Lactuca sativa plant or cell.

Techniques for introducing DNA into plant cells or plants are well-known to those of skill in the art. Four basic methods for delivering foreign DNA into plant cells or plants have been described. Chemical methods (see e.g, Graham and van der Eb, Virology, 54 (02): 536-539, 1973; Zatloukal, Wagner, Cotten, Phillips, Plank, Steinlein, Curiel, Birnstiel, Ann. N. Y. Acad. Sci., 660: 136-153, 1992); Physical methods including microinjection (see e.g., Capecchi, Cell, 22 (2): 479-488, 1980), electroporation (see e.g., Wong and Neumann, Biochim. Biophys. Res. Conmmun. 107 (2): 584-587, 1982; Fromm, Taylor, Walbot, Proc. Natl. Acad. Sci. USA, 82 (17): 5824-5828, 1985; U.S. Pat. No. 5,384,253) and the gene gun (see e.g., Johnston and Tang, Methods Cell. Biol., 43 (A): 353-365, 1994; Fynan, Webster, Fuller, Haynes, Santoro, Robinson, Proc. Natl. Acad. Sci. USA 90 (24): 11478-11482, 1993); Viral methods (see e.g., Clapp, Clin. Perinatol., 20 (1): 155-168, 1993; Lu, Xiao, Clapp, Li, Broxmeyer, J. Exp. Med. 178 (6): 2089-2096, 1993; Eglitis and Anderson, Biotechniques, 6 (7): 608-614, 1988; Eglitis, Kantoff, Kohn, Karson, Moen, Lothrop, Blaese, Anderson, Avd. Exp. Med. Biol., 241: 19-27, 1988); and Receptor-mediated methods (see e.g., Curiel, Agarwal, Wagner, Cotten, Proc. Natl. Acad. Sci. USA, 88 (19): 8850-8854, 1991; Curiel, Wagner, Cotten, Birnstiel, Agarwal, Li, Loechel, Hu, Hum. Gen. Ther., 3 (2): 147-154, 1992; Wagner et al., Proc. Natl. Acad. Sci. USA, 89 (13): 6099-6103, 1992).

The introduction of DNA into plant cells or plant by means of electroporation is well-known to those of skill in the art. Plant cell wall-degrading enzymes, such as pectin-degrading enzymes, are used to render the recipient cells more susceptible to transformation by electroporation than untreated cells. To effect transformation by electroporation one may employ either friable tissues such as a suspension culture of cells, or embryogenic callus, or immature embryos or other organized tissues directly. It is generally necessary to partially degrade the cell walls of the target plant material to pectin-degrading enzymes or mechanically wounding in a controlled manner. Such treated plant material is ready to receive foreign DNA by electroporation.

Another method for delivering foreign DNA to plant cells or plant is by microprojectile bombardment. In this method, microparticles are coated with foreign DNA and delivered into cells by a propelling force. Such micro particles are typically made of tungsten, gold, platinum, and similar metals. An advantage of microprojectile bombardment is that neither the isolation of protoplasts (Cristou et al., 1988, Plant Physiol., 87: 671-674) nor the susceptibility to Agrobacterium infection is required. An illustrative embodiment of a method for delivering DNA into maize cells by acceleration is a Biolistics Particle Delivery System, which can be used to propel particles coated with DNA or cells through a screen onto a filter surface covered with corn cells cultured in suspension. The screen disperses the particles so that they are not delivered to the recipient cells in large aggregates. For the bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of the microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids.

Agrobacterium-mediated transfer is a widely applicable system for introducing foreign DNA into plant cells because the DNA can be introduced into whole plant tissues, eliminating the need to regenerate an intact plant from a protoplast. The use of Agrobacterium-mediated plant integrating vectors to introduce DNA into plant cells is well known in the art. See, for example, the methods described in Fraley et al., 1985, Biotechnology, 3: 629; Rogers et al., 1987, Meth. in Enzymol., 153: 253-277. Further, the integration of the Ti-DNA is a relatively precise process resulting in few rearrangements. The region of DNA to be transferred is defined by the border sequences, and intervening DNA is usually inserted into the plant genome as described in Spielmann et al., 1986, Mol. Gen. Genet., 205: 34; Jorgensen et al., 1987, Mol. Gen. Genet., 207: 471.

Modern Agrobacterium transformation vectors are capable of replication in E. coli as well as Agrobacterium, allowing for convenient manipulations. Moreover, recent technological advances in vectors for Agrobacterium-mediated gene transfer have improved the arrangement of genes and restriction sites in the vectors to facilitate construction of vectors capable of expressing various proteins or polypeptides. Convenient multi-linker regions flanked by a promoter and a polyadenylation site for direct expression of inserted polypeptide coding genes are suitable for present purposes. In addition, Agrobacterium containing both armed and disarmed Ti genes can be used for the transformations.

Transformation of plant protoplasts can be achieved using methods based on calcium phosphate precipitation, polyethylene glycol treatment, electroporation, and combinations of these treatments (see, e. g., Potrykus et al., 1985, Mol. Gen. Genet., 199: 183; Marcotte et al., Nature, 335: 454, 1988).

Techniques for introducing DNA into a plastid are also well-known to those of skill in the art. For example, foreign DNA may be introduced into a plastid using a biolistic bombardment method, such as a method using a PDS-1000/He Particle Delivery System and following the modified protocol from Verma et al., 2008.

Once the recombinant DNA molecule has been introduced, the transgenic plastid, transgenic plant cell or transgenic plant may be cultivated or regenerated. Methods for cultivation or regenerating numerous plant species have been reported in the literature and are well known to the skilled artisan.

The present invention also provides methods of producing a tetravalent chimeric dengue virus antigen. Particularly, the present invention provides a method of producing a tetravalent chimeric dengue virus antigen comprising the steps of a) producing a transgenic plant as detailed herein, and b) incubating said plant under conditions wherein said plant expresses said antigen.

### Vaccines

The present invention also provides a vaccine. Particularly, the present invention provides a vaccine comprising a transgenic plant cell, a transgenic plant or a transgenic part of said plant as detailed herein optionally together with one or more pharmaceutically acceptable excipients. The present invention also provides transgenic plant cells, transgenic plants or transgenic parts of said plants as detailed herein for use in vaccination, and more for use in vaccinating a subject. Also provided is a method of vaccinating a subject, such as a human, against dengue virus, the method comprising the step of administering an effective amount of a vaccine as detailed herein to said subject. Also contemplated by the present invention is the use of a transgenic plant cell, a transgenic plant or a transgenic part of said plant as detailed herein in the manufacture of a medicinal product. Particularly, the present invention provides the use of a transgenic plant cell, a transgenic plant or a transgenic part of said plant as detailed herein in the manufacture of a medicinal product for vaccination of a subject, such as a human, against dengue virus. A medicinal product according to the present invention may be a pharmaceutical composition, and more particularly a vaccine.

The present invention thus provides means for protecting a subject against an dengue virus infection, and more particularly protects a subject against dengue fever (DF), dengue haemorrhagic fever (DHF) and/or dengue shock syndrome (DSS).

It is understood that the details given below for a particular aspect, such as the vaccine of the invention, applies mutatis mutandis to the other aspects mentioned above.

A vaccine according to the present invention may be administered orally. This administration route ensures inducing a mucosal immune response as well as takes advantage of cost and convenience. Conveniently, an oral administration entails consuming a transgenic plant or plant part according to the invention. For the purpose of vaccination, the transgenic plant or plant part according to the invention may be consumes in raw form. the transgenic plant or plant part according to the invention may however be formulated in the vaccine in a proceed from.

A vaccine according to the invention can be in the form of a plant part, an extract, a juice, a liquid, a powder or a tablet. According to particular embodiments, the vaccine is in the form of a liquid.

A vaccine according to the present invention may comprise one or more pharmaceutically acceptable excipients. The vaccine may be formulated using pharmaceutically acceptable carriers well known in the art in dosages suitable for oral administration. Such carriers enable the vaccine to be formulated as tablets, pills, dragees, capsules, liquids, gels, syrups, slurries, suspensions and the like, for ingestion by the subject (e.g., a human). A physiologically compatible carrier may be an physiologically acceptable diluent such as water, phosphate buffered saline, or saline.

A vaccine according to the present invention may comprise an adjuvant. Adjuvants such as incomplete Freund's adjuvant, aluminum phosphate, aluminum hydroxide, or alum are materials well known in the art.

A vaccine for oral use can be obtained through combination of the active ingredient with solid excipient, optionally grinding a resulting mixture, and processing the mixture of granules, after adding suitable auxiliaries, if desired, to obtain tablets or dragee cores.

Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Generally, the vaccine according to the present invention is capable of eliciting an immune response upon administration to a subject. Vaccines for use according to the present invention normally contain a therapeutically effective amount to the active ingredient (i.e., the polyprotein as detailed herein). The determination of an effective dose is well within the capability of those skilled in the art. The therapeutically effective dose can be estimated initially either in cell culture assays, or in animal models, usually birds, mice, rabbits, dogs, or pigs. The animal model is also used to achieve a desirable concentration range. Such information can then be use to determine useful doses in humans.

Dosage amounts may vary from 0.1 to 500,000 micrograms of recombinant polyprotein; transgenic plant cell, or transgenic plant per subject per day, for example, 1 μg, 10 μg, 100 μg, 500 μg, 1 mg, 10 mg, 100 mg, 1 g, 10 g, 50 g, 100 g, 200 g, and even up to a total dose of about 500 g per subject per day. According to certain embodiments, the dosage is in the range of 1 ng to 5 g per kilogram bodyweight. According to another certain embodiments, the dosage is in the range of 1 μg to 5 g per kilogram bodyweight. According to other certain embodiments, the dosage is in the range of 0.1 to 5 g per kilogram bodyweight. According to other certain embodiments, the dosage is in the range of 1 to 5 mg per kg body weight.

The present invention also provides methods of producing a vaccine. Particularly, the present invention provides a method of producing a vaccine comprising the steps of a) Producing a transgenic plant cell, a transgenic plant or a transgenic part of said plant comprising a recombinant DNA molecule as detailed herein, b) incubating said transgenic plant cell, said transgenic plant or said transgenic part of said plant under conditions wherein said plant cell, plant or part of said plant expresses the polyprotein encoded by said recombinant DNA molecule, c) harvesting said transgenic plant cell, said transgenic plant or said transgenic part of said plant, and d) formulating said transgenic plant cell, said transgenic plant or said transgenic part of said plant into a vaccine, optionally together with one or more pharmaceutically acceptable excipients.

It is also understood that the details given above with respect to the vaccine of the invention, applies mutatis mutandis to this aspect.

The transgenic plant cell, the transgenic plant or transgenic part of said plant may be formulated in its raw form. Alternatively, the transgenic plant cell, the transgenic plant or transgenic part of said plant may be formulated in a processed form, such as in the form of an extract or juice.

### CERTAIN DEFINITIONS

As used herein, “immune response” refers to a response made by the immune system of an organism to a substance, which includes but is not limited to foreign or self proteins. There are three general types of “immune response” including, but not limited to mucosal, humoral and cellular” immune responses. A “mucosal immune response results from the production of secretory IgA (sIgA) antibodies in secretions that bathe all mucosal surfaces of the respiratory tract, gastrointestinal tract and the genitourinary tract and in secretions from all secretory glands (McGhee, J. R. et al., 1983, Annals NY Acad. Sci. 409). These sIgA antibodies act to prevent colonization of pathogens on a mucosal surface (Williams, R. C. et al., Science 177, 697 (1972); McNabb, P. C. et al., Ann. Rev. Microbiol. 35, 477 (1981)) and thus act as a first line of defense to prevent colonization or invasion through a mucosal surface. The production of sIgA can be stimulated either by local immunization of the secretory gland or tissue or by presentation of an antigen to either the gut-associated lymphoid tissue (GALT or Peyer's patches) or the bronchial-associated lymphoid tissue (BALT; Cebra, J. J. et al., Cold Spring Harbor Symp. Quant. Biol. 41, 210 (1976); Bienenstock, J. M., Adv. Exp. Med. Biol. 107, 53 (1978); Weisz-Carrington, P. et al., J. Immunol 123, 1705 (1979); McCaughan, G. et al., Internal Rev. Physiol 28, 131 (1983)).

Membranous microfold cells, otherwise known as M cells, cover the surface of the GALT and BALT and may be associated with other secretory mucosal surfaces. M cells act to sample antigens from the luminal space adjacent to the mucosal surface and transfer such antigens to antigen-presenting cells (dendritic cells and macrophages), which in turn present the antigen to a T lymphocyte (in the case of T-dependent antigens), which process the antigen for presentation to a committed B cell. B cells are then stimulated to proliferate, migrate and ultimately be transformed into an antibody-secreting plasma cell producing IgA against the presented antigen.

When the antigen is taken up by M cells overlying the GALT and BALT, a generalized mucosal immunity results with sIgA against the antigen being produced by all secretory tissues in the body (Cebra et al., supra; Bienenstock et al., supra; Weinz-Carrington et al., supra; McCaughan et al., supra). Oral immunization is therefore an important route to stimulate a generalized mucosal immune response and, in addition, leads to local stimulation of a secretory immune response in the oral cavity and in the gastrointestinal tract.

An “immune response” may be measured using techniques known to those of skill in the art. For example, serum, blood or other secretions may be obtained from an organism for which an “immune response” is suspected to be present, and assayed for the presence of the above mentioned immunoglobulins using an enzyme-linked immuno-absorbant assay (ELISA; U.S. Pat. No. 5,951,988; Ausubel et al., Short Protocols in Molecular Biology 3rd Ed. John Wiley & Sons, Inc. 1995). According to the present invention, a vaccine of the present invention can be said to stimulate an “immune response” if the quantitative measure of immunoglobulins in an subject treated with a vaccine of the present invention detected by ELISA is statistically different (for example, is increased by 2-fold or more, for example, 2, 3, 4, 5, 10, 20, 30, 40, 50, 60, 70, 80, 90, 100, or 1000-fold or more increase or decrease in the amount of antibody produced. An increase also means at least 5% or more antibody production, for example, 5, 6, 10, 20, 30, 40, 50, 60 70, 80, 90 or 100% or more from the measure of immunoglobulins detected in a subject not treated with the vaccine, wherein said immunoglobulins are specific for the polyprotein of the present invention. A statistical test known in the art and useful to determining the difference in measured immunoglobulin levels includes, but is not limited to ANOVA, Student's T-test, and the like, wherein the P value is at least <0.1, <0.05, <0.01, <0.005, <0.001, and even <0.0001.

An “immune response” may be measured using other techniques such as immunohistochemistry using labeled antibodies which are specific for portions of the immunoglobulins raised during the “immune response”. Tissue (e.g., ovarian tissue) from a subject to which a vaccine has been administered according to the invention may be obtained and processed for immunohistochemistry using techniques well known in the art (Ausubel et al., Short Protocols in Molecular Biology 3rd Ed. John Wiley & Sons, Inc. 1995). Microscopic data obtained by immunohistochemistry may be quantitated by scanning the immunohistochemically stained tissue sample and quantitating the level of staining using a computer software program known to those of skill in the art including, but not limited to NIH Image (National Institutes of Health, Bethesda, Md.).

As used herein, “subject” refers to an organism classified within the phylogenetic kingdom animalia. As used herein, an “subject” also refers to a mammal, and in particular a human.

As used herein, “monocotyledonous” refers to a type of plant whose embryos have one cotyledon or seed leaf. Examples of “monocots” include, but are not limited to lilies; grasses; corn; grains, including oats, wheat and barley; orchids; irises; onions and palms.

As used herein, “dicotyledonous” refers to a type of plant whose embryos have two seed halves or cotyledons. Examples of “dicots” include, but are not limited to lettuce, tobacco, tomato, alfalfa, mints; and walnuts.

As used herein, “vector” refers to a nucleic acid molecule capable of transporting another nucleic acid molecule to which it has been linked. One type of vector is a “plasmid”, which refers to a circular double stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of a recombinant DNA molecule into a genome of a plant. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

As used herein, “promoter” refers to a sequence of DNA, usually upstream (5′) of the coding region of a structural gene, which controls the expression of the coding region by providing recognition and binding sites for RNA polymerase and other factors which may be required for initiation of transcription. The selection of the promoter will depend upon the nucleic acid sequence of interest. A “promoter functional in a plant cell” refers to a “promoter” which is capable of supporting the initiation of transcription in plant cells, causing the production of an mRNA molecule.

As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. A promoter sequence is “operably-linked” to a gene when it is in sufficient proximity to the transcription start site of a gene to regulate transcription of the gene.

“Administering” or “administer” is defined as the introduction of a substance, such as the transgenic plant cell, transgenic plant, transgenic part of a transgenic plant and vaccines of the present invention, into the body of a subject and includes mucosal, oral, nasal, rectal, vaginal and parenteral routes. Particular routes of administration are mucosal routes, and more particular the oral route.

As used herein, “pharmaceutically acceptable” means a non-toxic material that does not interfere with the effectiveness of the biological activity of the active ingredient(s).

As used herein, a “carrier” refers to an inert and non-toxic material suitable for accomplishing or enhancing delivery of the vaccine of the present invention into a subject.

As used herein, “incubating” includes growing a plant either in the field or in a controlled or uncontrolled laboratory or indoor setting.

“% Identity” of two nucleotide sequences refers to sequence identity between a nucleotide sequence and a reference nucleotide sequence. Identity can be determined by comparing a position in each sequence which may be aligned for purposes of comparison. When a position in the compared sequences is occupied by the same base, then the molecules are identical at that position. A degree of similarity or identity between nucleotide sequences is a function of the number of identical or matching nucleotides at positions shared by the nucleotide sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.

“% identity” of an amino acid sequence to a reference amino acid sequence, as used herein, defines the % identity calculated from the two amino acid sequences as follows: The sequences are aligned using Version 9 of the Genetic Computing Group's GAP (global alignment program), using the default BLOSUM62 matrix (see below) with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence.

The following BLOSUM62 matrix is used:

| | | | | | | | | | | | | | | | | | | | | |
|---|---|---|---|---|---|---|---|---|---|---|---|---|---|---|---|---|---|---|---|---|
| Ala | 4 | | | | | | | | | | | | | | | | | | | |
| Arg | −1 | 6 | | | | | | | | | | | | | | | | | | |
| Asn | −2 | 0 | 6 | | | | | | | | | | | | | | | | | |
| Asp | −2 | −2 | 1 | 6 | | | | | | | | | | | | | | | | |
| Cys | 0 | −3 | −3 | −3 | [symbol] | | | | | | | | | | | | | | | |
| Gln | −1 | 1 | 0 | 0 | −3 | [symbol] | | | | | | | | | | | | | | |
| Glu | −1 | 0 | 0 | 2 | −4 | [symbol] | 5 | | | | | | | | | | | | | |
| Gly | 0 | −2 | 0 | −1 | −3 | −2 | −2 | 6 | | | | | | | | | | | | |
| His | −2 | 0 | 1 | −1 | −3 | 0 | 0 | [symbol] | [symbol] | | | | | | | | | | | |
| Ile | −1 | −3 | −3 | −3 | −1 | −3 | −3 | −4 | −3 | [symbol] | | | | | | | | | | |
| Leu | −1 | −2 | −3 | −4 | −1 | −2 | −3 | −4 | −3 | 2 | 4 | | | | | | | | | |
| Lys | −1 | 2 | 0 | −1 | −3 | 1 | 1 | −2 | −1 | −3 | −2 | [symbol] | | | | | | | | |
| Met | −1 | −1 | −2 | −3 | −1 | 0 | −2 | −3 | −2 | 1 | 2 | −1 | 5 | | | | | | | |
| Phe | −2 | −3 | −3 | −3 | −2 | −3 | −3 | −3 | −1 | 0 | 0 | −3 | 0 | 6 | | | | | | |
| Pro | −1 | −2 | −2 | −1 | −3 | −1 | −1 | −2 | −2 | −3 | −3 | −1 | −2 | −4 | 7 | | | | | |
| Ser | 1 | −1 | 1 | 0 | −1 | 0 | 0 | 0 | −1 | −2 | −2 | 0 | −1 | −2 | −1 | 4 | | | | |
| Thr | 0 | −1 | 0 | −1 | −1 | −1 | −1 | −2 | −2 | −1 | −1 | −1 | −1 | −2 | −1 | 1 | 6 | | | |
| Trp | −3 | −3 | −4 | −4 | −2 | −2 | −3 | −2 | −2 | −3 | −2 | −3 | −1 | 1 | −4 | −3 | −2 | 11 | | |
| Tyr | −2 | −2 | −2 | −3 | −2 | −1 | −2 | −3 | 2 | −1 | −1 | −2 | −1 | 3 | −3 | −2 | −2 | 2 | 7 | |
| Val | 0 | −3 | −3 | −3 | −1 | −2 | −2 | −3 | −3 | 3 | 1 | −2 | 1 | −1 | −2 | −2 | 0 | −3 | −1 | 4 |
| | Ala | Arg | Asn | Asp | Cys | Gln | Glu | Gly | His | Ile | Leu | Lys | Met | Phe | Pro | Ser | Thr | Trp | Tyr | Val |

[symbol]

indicates data missing or illegible when filed

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### Examples

### Materials

### 1. Bacterial Strains

One Shot® OmniMAX™ 2 T1^{R} chemically competent E. coli (Cat. No. C8540-03, Invitrogen™, Thermo Fisher Scientific Inc., USA)

One Shot® ccdB Survival™ 2 T1^{R} chemically competent E. coli (Cat. No. A10460. Invitrogen™, Thermo Fisher Scientific Inc., USA)

### 2. Plant Materials

Seeds of Lactuca sativa L. cv. Barkley: (WT)

Constructed transplastomic plant lines:

1) Lactuca sativa line expressing EDIII 1-4 constitutive: S12-PN-EDIII 1-4
2) Lactuca sative line expressing EDIII 1 constitutive: S16-PN-EDIII 1

### 3. Appliances and Consumables

TABLE 1
Ready to use reagents and Kits.

| | Company |
|---|---|
| Kits | |
| AP-conjugate substrate Kit, Cat. No. 170-6432 | Bio-Rad, USA |
| DIG-High Prime DNA Labeling and Detection Starter | Roche Applied Science, USA |
| Kit II; Kit for chemiluminescent detection with CSPD, | |
| Cat. No. 11585614910 | |
| Gateway ® BP Clonase ® Enzyme Mix, Cat. No. | Invitrogen, USA |
| 11789-013 | |
| Gateway ® LR Clonase ® Enzyme Mix, Cat. No. | Invitrogen, USA |
| 11791019 | |
| Gateway ® Vector Conversion System with One | Invitrogen, USA |
| Shot ® ccdB Survival Cells, Cat. No. 11828-029 | |
| HisPur ™ Cobalt Purification Kit, Cat. No. 90090 | Thermo Scientific, USA |
| iBlot ® Gel Transfer Stacks Nitrocellulose | Life technologies, USA |
| Pierce ™ BCA Protein Assay Kit, Cat. No. 23227 | Thermo Scientific, USA |
| Qiagen ® Plasmid Maxi Kit, Cat. No. 122637 | Qiagen GmbH, Germany |
| Qiagen ® Plasmid Midi Kit, Cat. No. 12145 | Qiagen GmbH, Germany |
| QIAprep ® Spin Miniprep Kit, Cat. No. 27106 | Qiagen GmbH, Germany |
| QIAquick ® Gel Extraction Kit, Cat. No. 28706 | Qiagen GmbH, Germany |
| Rapid DNA ligation Kit, Cat. No. 1422 | Thermo Scientific, USA |
| Reagents | |
| 0.6 μm Gold Microcarriers | Bio-Rad, USA |
| 1 kb DNA ladder | New England Biolabs, USA |
| 100 bp DNA ladder | New England Biolabs, USA |
| 100 mM dNTP Set, PCR Grade | Invitrogen, USA |
| 6X Loading Dye | Thermo Scientific, USA |
| Anti-Rabbit IgG (Fc), AP conjugated, Cat. No. S3731 | Promega, USA |
| Ethidium bromide 1% in water | Carl Roth, Germany |
| GeneAmp ® 10X PCR Buffer | Applied Biosystems, USA |
| Lambda DNA/Eco130I (Styl) Marker 16 | Fermentas, Lithuania |
| Polyclonal rabbit anti-dengue antibody produced | Davids Biotechnology, Germany |
| against amino acid sequence: KFKVVKEIAETQHGT | |
| (SEQ ID NO: 11) | |
| Polyclonal rabbit anti-GFP antibody, Cat. No. | antibodies-online GmbH, |
| ABIN398856 | Germany |
| Protein marker IV | Peqlab GmbH, Germany |
| Quick Start ™ Bovine Serum Albumin Standard | Bio-Rad, USA |
| Quick Start ™ Bradford 1x Dye Reagent | Bio-Rad, USA |
| Sigmafast ™ BCIP ®/NBT | Sigma-Aldrich, USA |
| Spectra Multicolor Broad Range Protein Ladder | Thermo Scientific, USA |

TABLE 2
Primers and probes.

| Primer | Sequence 5′-3′ | | | | | SEQ ID NO: | |
|---|---|---|---|---|---|---|---|
| p1 | ACCCATGGCTTCTAAAGGAG | | | | | 12 | |
| p2 | AGACAGCGACGGGTTCTCTG | | | | | 13 | |
| 103 | GATCCGAGCCATAGAATTTC | | | | | 14 | |
| p4 | TGCTGGCCGTACATTTGTACG | | | | | 15 | |
| P5 | TACCCGGGAATTGTGACCTC | | | | | 16 | |
| p6 | AGAGTCCGACCACAACGACC | | | | | 17 | |
| p7 | GCTGAAACTCAACATGGAACTG | | | | | 18 | |
| p8 | ATGCTTTTTCACCAGCACCT | | | | | 19 | |
| P9 | TTGCTGAAACTCAACATGGA | | | | | 20 | |
| p10 | CCAAAAGGAGGTTCAGCTTC | | | | | 21 | |
| p11 | TGAAGATGGACAAGGAAAAGC | | | | | 22 | |
| p12 | CTCCACCACCTCCTTTACCA | | | | | 23 | |
| p13 | ACTACTCAAGCTGCATTATATACC | | | | | 24 | |
| p14 | GCACCTTTTACTAAGATCAATG | | | | | 25 | |
| p15 | GGAGGTAGGATGGGCAGTTG | | | | | 26 | |
| p16 | GGACTCGAACCGCTGACATC | | | | | 27 | |
| p17 | GGACTCGAACCGCTGACATC | | | | | 28 | |
| p18 | AACGACCTTTTGGAAACTTC | | | | | 29 | |
| p19 | TCTGTGAGCGTGACGGTGGT | | | | | 30 | |
| p20 | TTACGCGAACGCGAAGTCCG | | | | | 31 | |
| pM13F | GTAAAACGACGGCCAG | | | | | 32 | |
| pM13R | CAGGAAACAGCTATGACC | | | | | 33 | |
| p296 | TGACTTATATACTCGTGTCAAC | | | | | 34 | |
| p297 | CTGCTAATGTCTACTGTTTGT | | | | | 35 | |
| Probe | aadA | den1 | den2 | den3 | insl | psaB | trnA |

Primers are custom-synthesized by Eurofins MWG Operon (Germany) and reconstituted with sterile ddH2O to a stock concentration of 100 mM, further diluted to a working concentration of 10 mM and stored at −20° C. in aliquots of 500 μl.

TABLE 3
Enzmyes.

| Name/Description | Company |
|---|---|
| AmpliTaq ® DNA Polymerase (5 U/μl) | Applied Biosystems, USA |
| ApaI, BglII, SmaI, NcoI, SaclI, PstI, | New England Biolabs, USA |
| EcoRV, XbaI, NheI, KpnI | |

TABLE 4
Chemicals.

| Name/Description | Company |
|---|---|
| Acetic acid (CH₃COOH) | Sigma-Aldrich, USA |
| Acrylamide/bis-acrylamide (19:1) 40% | Sigma-Aldrich, USA |
| Agarose | Sigma-Aldrich, USA |
| Ammonium acetate (NH₄Ac) | Sigma-Aldrich, USA |
| Ammoniumpersulfate (APS) | Sigma-Aldrich, USA |
| Ampicillin | Sigma-Aldrich, USA |
| Bacto Agar | Carl Roth, Germany |
| Bacto Tryptone | Carl Roth, Germany |
| BAP (6-benzylaminopurine) | Sigma-Aldrich, USA |
| Bovine Serum Albumin (BSA) | Sigma-Aldrich, USA |
| Brilliant Blue G | Sigma-Aldrich, USA |
| Calcium Chloride Dihydrate | Merck, Germany |
| Chloramphenicol | Duchefa Biochemie, Netherlands |
| Chloroform | Sigma-Aldrich, USA |
| cOmplete Protease Inhibitor Cocktail | Roche Applied Science, USA |
| Developer | AGFA, Belgium |
| Dithiothreitol (DTT) | Sigma-Aldrich, USA |
| EDTA dinatriumsalt | Fluka, Switzerland |
| Ethanol 96% | Sigma-Aldrich, USA |
| Fixer | AGFA, Belgium |
| Gelzan ™ CM Gelrite ® | Sigma-Aldrich, USA |
| Glycerol | Carl Roth, Germany |
| Glycine | Carl Roth, Germany |
| Hexadecyl-trimethyl- | Duchefa Biochemie, Netherlands |
| ammonium bromide (CTAB) | |
| Hydrochloric Acid (HCl) | J. T. Baker, Netherlands |
| Isopropanol | Merck, Germany |
| Kanamycin sulfate | Sigma-Aldrich, USA |
| Magnesium acetate tetrahydrate | Sigma-Aldrich, USA |
| Magnesium chloride hexahydrate | Carl Roth, Germany |
| Maleic acid | Fluka, Switzerland |
| Methanol | Fluka, Switzerland |
| MS (Murashig&Skoog) incl. vitamins | Duchefa Biochemie, Netherlands |
| N,N,N′,N′- | Sigma-Aldrich, USA |
| tetramethylethylenediamine (Temed) | |
| Naphtalene acetic acid (NAA) | Duchefa Biochemie, Netherlands |
| Polyvinylpyrrolidone (PVP) 40 | Sigma-Aldrich, USA |
| Potassium acetate (KAc) | Carl Roth, Germany |
| Potassium chloride (KCl) | Sigma-Aldrich, USA |
| Potassium hydroxide (KOH) | Merck, Darmstadt, Germany |
| Potassium phosphate | Sigma-Aldrich, USA |
| monobasic (KH₂PO₄) | |
| SDS (Sodium lauryl sulfate) | Sigma-Aldrich, USA |
| Sodium acetate (NaC2H3O2) | Merck, Germany |
| Sodium Chloride (NaCl) | Carl Roth, Germany |
| Sodium Citrate tribasic dihydrate | Sigma-Aldrich, USA |
| Sodium hydroxide (NaOH) | Carl Roth, Germany |
| Sodium phosphate dibasic (Na2HPO4) | Sigma-Aldrich, USA |
| Spectinomycin dihydrochloride | Sigma-Aldrich, USA |
| Spermidine | Duchefa Biochemie, Netherlands |
| β-Mercaptoethanol | Sigma-Aldrich, USA |
| Sucrose | Duchefa Biochemie, Netherlands |
| Tris | Duchefa Biochemie, Netherlands |
| Tween 20 | Sigma-Aldrich, USA |
| Yeast extract | Carl Roth, Germany |

TABLE 5
Buffers, Solutions and Media.

| Name/Description | Recipe | | |
|---|---|---|---|
| Buffers | | | |
| Alkali-Transfer | | | Concentration |
| Buffer 5X, 1 L | 175.5 g | NaCl | 3M |
| | 80 g | NaOH | 2M |
| | 1 L | H₂O | |
| AP color | AP color development Buffer 25X was diluted 1:25 in | | |
| development | distilled water end stored at +4° C. | | |
| Buffer 1X | | | |
| Blotting Buffer, | | | Concentration |
| 10X, pH 8.3 | 3.02 g | Tris | 25 mM |
| for 1 L | 14.4 g | Glycine | 192 mM |
| | 200 ml | Methanol | 20% |
| | 1 L | H₂O | |
| CTAB Buffer | | | Concentration |
| 1X, 200 ml | 4 g | CTAB | 2% |
| | 40 ml | 1M Tris-HCl | 200 mM |
| | 8 ml | 0.5M EDTA | 20 mM |
| | 16.36 g | NaCl | 1.4M |
| | 2 g | PVP 40 | 1% |
| | filled to 200 ml with distilled water, sterilized by | | |
| | autoclaving and stored at +4° C. | | |
| Detection Buffer | | | Concentration |
| 1X, pH 9.5, | 3 ml | 1M Tris HCL | 0.1M |
| 30 ml | 60 μl | 5M NaCl | 0.1M |
| | 27 ml | H₂O | |
| Laemmli Buffer | | | Concentration |
| (LB) 5X, | 5 g | SDS | 10% |
| pH 6.8, 50 ml | 5 ml | 13M β-MercaptoEtOH | 10% |
| | 25 ml | 100% Glycerol | 50% |
| | 45 μl | 100% Bromphenol Blue | 0.09% |
| | 10.42 ml | 1.5M Tris-HCl, pH 6.8 | 312.5 mM |
| | 10 ml | H₂O | |
| Maleic acid | | | Concentration |
| Buffer 1X | 100 ml | Maleic, acid 10X | 0.1M |
| pH 7.5, 1 L | 100 ml | 1M NaCl | 0.1M |
| | 800 ml | H₂O | |
| PBS 10X, pH | | | Concentration |
| 7.4, 1 L | 80 g | NaCl | 137 mM |
| | 2 g | KCl | 2.7 mM |
| | 14.4 g | Na₂HPO₄ | 100 mM |
| | 2.4 g | KH₂PO₄ | 2 mM |
| | Filled to 1 L with distilled water and 6utoclaved. | | |
| PBS-T 1X | PBS 10X was diluted 1:10 in distilled water; 1 ml | | |
| | Tween-20 is added/L buffer. | | |
| Plant Extraction | | | Concentration |
| Buffer I (PEB I), | 200 μl 5M NaCl | | 100 mM |
| 1X, 10 ml | 200 μl 0.5M EDTA, pH 8 | | 10 mM |
| | 2 ml 1M Tris-HCl, pH 8 | | 200 mM |
| | 5 μl Tween-20 | | 0.05% |
| | 100 μl 10% SDS | | 0.1% |
| | 11 μl 13M β-MercaptoEtOH | | 14 mM |
| | 2 ml 1M Sucrose | | 200 mM |
| | 200 μl 100 mM PMSF | | 2 mM |
| | 5.3 ml H₂O | | |
| Plant Extraction | | | Concentration |
| Buffer II | 500 μl 1M Hepes-KOH pH 7.5 | | 50 mM |
| (PEB II), | 20 μl 0.5M EDTA pH 8 | | 1 mM |
| 1X, 10 ml | 20 ml 5M Kac | | 10 mM |
| | 500 μl 0.1M MgAc | | 5 mM |
| | 10 μl 1M DTT | | 1 mM |
| | 1 cOmplete mini tablet | | 1X |
| | 8.7 ml H₂O | | |
| Pant Extraction | | | Concentration |
| Buffer III | 714 μl 1M Sucrose | | 0.7M |
| (PEB III), | 5 ml 1M Tris-HCl pH 9.5 | | 0.5M |
| 1X, 10 ml | 1 ml 0.5M EDTA | | 50 mM |
| | 833 μl 1.2M KCl | | 0.1M |
| | 20 μl 13M β-MercaptoEtOH | | 0.2% |
| | 1 cOmplete mini tablet | | 1X |
| | 8.7 ml H₂O | | |
| Running Buffer, | | | Concentration |
| 10X, pH 8.3, | 30.2 g | Tris | 0.25M |
| 1 L | 144 g | Glycine | 1.92M |
| | 10 g | SDS | 1% |
| | 1 L | H₂O | |
| Separating Gel- | | | Concentration |
| Buffer 4X, | 181.5 g | Tris | 1.5M |
| pH 8.8, 1 L | 2 g | SDS | 0.2% |
| | 1 L | H₂O | |
| | Set pH with concentrated HCl | | |
| Stacking Gel- | | | Concentration |
| Buffer 4X, | 60.5 g | Tris | 0.5M |
| pH 6.8, 1 L | 2 g | SDS | 0.2% |
| | 1 L | H₂O | |
| | Set pH with concentrated HCl | | |
| TAE Buffer 1X, | TAE 50X was diluted 1:50 in distilled water. | | |
| 1 L | | | |
| TAE Buffer | | | Concentration |
| 50X, 1 L | 242 g | Tris | 2M |
| | 7.1 ml | Acetic acid | 0.7% |
| | 100 ml | 0.5M EDTA | 0.05M |
| | Filled to 1 L with distilled water and autoclaved. | | |
| TBS 10X, | | | Concentration |
| pH 7.6, 1 L | 80 g | NaCl | 1370 mM |
| | 24.2 g | Tris | 200 mM |
| | Filled to 1 L with distilled water and autoclaved. | | |
| TBS-T 1X | TBS 10X was diluted 1:10 in distilled water, | | |
| | 1 ml Tween 20 was added/L Buffer | | |
| TE Buffer 1X, pH | | | Concentration |
| 5.6, 100 ml | 1 ml | 1M Tris-HCl | 10 mM |
| | 200 μl | 0.5M EDTA | 1 mM |
| | Filled to 100 ml with distilled water and autoclaved. | | |
| Wash Buffer | | | Concentration |
| (WB) 1X, | | Maleic acid | |
| pH 7.5, 1 L | 750 ml | Buffer 1X | 1x |
| | 2 ml | Tween-20 | 0.3% |

| | |
|---|---|
| Solutions | |
| Acryl-amide 30% | 37 ml of Acryl-amide (40%) filled up to 50 ml with |
| | distilled water and stored at +4° C. |
| Ampicillin 25 | 250 mg ampicillin were dissolved in 10 ml distilled |
| mg/ml, 10 ml | water, filter sterilized and stored at −20° C. |
| APS 10% | 1 g Ammonium persulfate was dissolved in 10 ml |
| | distilled water, aliquoted and store at −20° C. |
| BAP 1 mg/ml, | 100 mg 6-benzylaminopurine were dissolved in 0.5 ml |
| 100 ml | 1M NaOH, filled up to 100 ml with distilled water, |
| | filter sterilized and stored at −20° C. |
| Blocking sotution | 10X Blocking solution was diluted 1:10 in |
| 1X for Southern | 1X Maleic acid Buffer |
| Blot | |
| Blocking solution | 0.5% BSA in 1X TBS-T or 1X PBS-7 |
| for Western | |
| Blot: | |
| CaCl₂ 2.5M, | 10.8 g CaCl₂ × 6H₂O were dissolved in |
| 20 ml | 20 ml distilled water, filter sterilized, aliquoted and |
| | stored at −20° C. |
| Chloramphenicol | 150 mg chloramphenicol were dissolved in 5 ml EtOH, |
| 3 mg/ml | filled up to 50 ml with distilled water, filter sterilized |
| | and stored at +4° C. |
| DTT 1M, 50 ml | 7.7 g DTT were dissolved in water and aliquots were |
| | stored at −20° C. |
| dNTPs 25 mM | 25 μl dATP |
| each, 100 μl | 25 μl dCTP |
| | 25 μl dGTP |
| | 25 μl dTTP |
| | stored in aliquots at −20° C. |
| EDTA 0.5M, pH | 84.05 g EDTA |
| 8.0, 500 ml | 11.25 g NaOH |
| | dissolved in 250 ml distilled water and autoclaved. |
| Kanamycin 25 | 250 mg kanamycin were dissolved in 100 ml distilled |
| mg/ml, 100 ml | water, filter sterilized and stored at −20° C. |
| Maleic acid 10X, | 116 g Maleic acid were dissolved in 1 L of distilled |
| pH 7.5, 1 L | water, pH adjusted with solid NaOH and autoclaved. |
| HCl 0.25M, 1 L | 25 ml 37% HCl were diluted with 975 ml distilled |
| | water. |
| MgAc 0.1M, | 10.7 g Magnesium acetate tetrahydrate were dissolved |
| 500 ml | in 500 ml distilled water and autoclaved. |
| NaCl 1M, 1 L | 58.44 g NaCl were dissolved in 1 L of distilled |
| | water and autoclaved. |
| NaCl 5M, 500 ml | 146.4 g NaCl were dissolved in 1 L of distilled |
| | water and autoclaved. |
| NaOH 0.4M, 1 L | 16 g NaOH were dissolved in 1 L distilled water. |
| NaAc 3M, pH | 49.2 g NaAc were dissolved in 200 ml distilled water. |
| 5.2, 200 ml | |
| 0.1M NH₄OAc, | 0.38 g Ammonium acetate were dissolved in 50 ml |
| 50 ml | Methanol and autoclaved. |
| KCl 250 mM, | 1.86 g KCl were dissolved in 100 ml distilled water. |
| 100 ml | |
| KAc 3M, 100 ml | 29.44 g KAc were dissolved in 100 ml distilled water. |
| pH 5.2 | |
| SDS 10%, 100 ml | 10 g SDS were added to 100 ml distilled water and |
| | heated at 68° C., pH was adjusted to 7.2 and sterilized |
| | by autoclaving. |
| Spectinomycin | 10 g Spectinomycin were dissolved in 100 ml distilled |
| (Spec) 100 | water, filter sterilized and stored at −20° C. |
| mg/ml, 100 ml | |
| Spermidine 0.1M, | 0.255 g Spermidine were dissolved in 10 ml distilled |
| 10 ml | water, aliquots were stored at −20° C. |
| SSC 0.5X + 0.1% | 12.5 ml 20X SSC and 5 ml 10% SDS were filled up to |
| SDS, 500 ml | 500 ml with distilled water. |
| SSC 2X + 0.1% | 50 ml 20X SSC and 5 ml 10% SDS were filled up |
| SDS, 500 ml | to 500 ml with distilled water. |
| Sucrose 1M, | 85.5 g Sucrose were dissolved in 250 ml distilled |
| 250 ml | water, autoclaved and stored at +4° C. |
| Tris-HCl 1M, 1 L | 121.1 g Tris were dissolved in 1000 ml distilled water, |
| | pH adjusted with concentrated HCl and autoclaved. |
| PMSF 100 mM, | 0.87 g PMSF were dissolved in 50 ml DMSO, |
| 50 ml | aliquots of 1 ml stored at −20° C. |
| Media | |
| LB-Medium, | 10 g Bacto Tryptone |
| pH 7.0, liquid, | 5 g Yeast extract |
| 1 L | 10 g NaCl |
| | Mixed and dissolved in 1 L of distilled water, adjusted |
| | pH to 7.0 and sterilized by autoclaving. |
| LB-Medium, | 10 g Bacto Tryptone |
| pH 7.0, solid, | 5 g Yeast extract |
| 1 L | 10 g NaCl |
| | 10 g Bacto-Agar |
| | Mixed and dissolved in 1 L of distilled water, adjusted |
| | pH to 7.0 and sterilized by autoclaving. |
| MS-medium, | 4.4 g MS-Salt including vitamins |
| pH 5.8, solid | 10 g Sucrose |
| 1 L | 3.1 g Gelzan ™ |
| | Mixed and dissolved in 1 L distilled water, adjusted |
| | pH to 5.8 and sterilized by autoclaving. |
| RMOP medium, | 4.4 g MS-Salt including vitamins |
| pH 5.8, solid, | 30 g Sucrose |
| 1 L | 3.1 g Gelzan ™ |
| | Filled up to 1 L with distilled water, adjusted to pH 5.8 |
| | and sterilized by autoclaving. After autoclaving the |
| | following is added: |
| | 1 ml BAP (1 mg/ml) |
| | 100 μl NAA (1 mg/ml) |
| | 5 ml Spec (100 mg/ml). |

TABLE 6
Laboratory equipment and material.

| Name/Description | Company |
|---|---|
| Appliances | |
| Bombardment chamber PDS1000He | Bio-Rad, USA |
| SP5 II confocal microscope system | Leica Microsystems, Germany |
| Autoclave | Matachana, Spain |
| Table Top Centrifuge | Thermo Scientific, USA |
| Clean bench Herasafe ™ | Thermo Scientific, USA |
| Vortex | VWR, USA |
| pH meter | WTW, Germany |
| Ultracentrifuge | Eppendorf, Germany |
| MJ Mini ™ Personal Thermo Cycler | Bio-Rad, USA |
| Shaker Titertek ® | Flow Laboratories, USA |
| Orbital Shaker SSL3 | Stuart, UK |
| Nanodrop | Thermo Scientific, USA |
| Retsch Mill | Retsch, Germany |
| Water bath | Grant, UK |
| Heat block | Techne Inc, USA |
| Hot plate stirrer | IKA-Works, USA |
| Precision Balance | Mettler-Toledo, Switzerland |
| Gel electrophorese apparatus | Bio-Rad, USA |
| Power supply | Bio-Rad, USA |
| iBlot Blotting device | Thermo Scientific, USA |
| PAA Electrophorese chamber | Bio-Rad, USA |
| Gel doc | Bio-Rad, USA |
| Minitron Incubator | Infors HT, Switzerland |
| Vacuum pump | Edwards Limited, UK |
| Fridge | Whirlpool, USA |
| Freezer −20° C. | Whirlpool, USA |
| Freezer −80° C. | Sanyo, Japan |
| Micropipets | Eppendorf, Germany |
| Glass plates for PAA Gels | Bio-Rad, USA |
| Spacers for PAA Gels | Bio-Rad, USA |
| Gel casting device for PAA Gels | Bio-Rad, USA |
| Macrocarrier | BioRad, USA |
| UV-VIS Spectrophotometer | Shimadzu, Japan |
| Multiplate Reader | Biochrom, UK |
| Milli-Q-water purification system | Merck-Millipore, Germany |
| HM-4000 Multidizer ™ Hybridization | UVP, USA |
| Oven | |
| Consumables | |
| glasware | Schott-Duran, Germany |
| 25/50 ml plastic tubes | Greiner Bio One, USA |
| Finntips 10 μl, 200 μl, 1000 μl | Thermo Scientifiy, USA |
| 1.5/2 ml reaction tubes | Eppendorf, Germany |
| PCR tubes | Thermo Scientific, USA |
| petridishes | VWR, USA |
| Magenta-boxes | Sigma-Aldrich, USA |
| Retsch mill steel beads | Retsch, Germany |
| Lumi Film for chemiluminescent detection | Roche Applied Science, USA |
| Roti ® plus nylon membrane 0.45 μm | Carl Roth, Germ |
| gloves | VWR, USA |
| Whatmann filter paper | GE Healthcare, UK |

### Methods

### 4. Bacterial Growth Conditions

Overnight cultivation of E. coli was done either on LB plates with 10 g/l Bacto-Agar or in 5 ml liquid LB medium containing the appropriate antibiotics (Table 7) for maintaining the selection pressure at 37° C. and shaking at 320 rpm. 33% glycerol stocks were made of every liquid culture by mixing 0.5 mL of 100% Glycerol and 1 mL liquid cell culture. The glycerol stocks were shock frozen in liquid N₂ and stored at −80° C.

TABLE 7
Final concentration of antibiotics in LB medium used
for plates and liquid culture of transformed E. coli.

| Antibiotic | Final concentration |
|---|---|
| ampicillin | 100 [mg/L] |
| chloramphenicol | 30 [mg/L] |
| kanamycin | 100 [mg/L] |
| spectinomycin | 500 [mg/L] for tobacco |
| | 30 [mg/L] for lettuce |

### 5. Plant Growth Conditions

### 5.1 Seed Sterilization

Seeds of Lactuca sativa were soaked with 5% Dimanin C along with few drops of washing liquid for 10 minutes. The solution was pipetted out and the seeds were then washed in 70% ethanol for 1 min, washed three times in distilled water and air dried at room temperature in the clean bench. The seeds were stored at 4° C.

### 5.2 In Vitro Plant Tissue Culture and Regeneration

Seeds were germinated on solid MS-Medium containing the appropriate antibiotic (Table 8); young seedlings were transferred to Magenta-Boxes containing the same medium.

Tissue culture of bombarded leave discs was carried out on RMOP-Medium containing the appropriate antibiotics (Table 8); young shoots developing from the callus tissue were transferred into Magenta-Boxes containing MS-medium incl. antibiotics for rooting and further growth.

All in vitro cultures were incubated at 25° C., at a 16 h light-8 h dark cycle in growth chambers equipped with Universal lamps with white fluorescence, light intensity; 0.5-1 W/m² Osram L85 W/25.

TABLE 8
Antibiotic concentrations used in medium for
seed germination, in vitro culture of plants and
tissue culture of bombarded leave discs.

| Seeds/plant | Antibiotic |
|---|---|
| L. sative | MS |
| S12-PN-EDIII 1-4 | MS + 30 mg/L spectinomycin |
| S16-PN-EDIII 1 | |
| Bombarded L. sativa leave discs | RMOP + 30 mg/L spectinomycin |

### 5.3 Greenhouse Growth Conditions

Rooted plants were transferred to soil and grown in the greenhouse with additional light for 16 h and light intensity of 300 μE·s⁻¹·m⁻², at 25° C. and relative humidity of 60%.

### 6. DNA Preparation

### 6.1 Plasmid DNA Isolation

Plasmid DNA was isolated using the Kits purchased from Qiagen (Qiagen® Plasmid Maxi Kit, Qiagen® Plasmid Midi Kit and QIAprep® Spin Miniprep Kit) and following the instructions supplied with the Kits.

Briefly, E. coli overnight culture in liquid LB medium was harvested by centrifugation at 8000 g for 3 min and the pellet was re-suspended in Buffer P1. The bacterial cell pellet was lysed by alkaline lysis, the cell debris and other contaminants were precipitated and the cleared lysate was applied to the silica membrane to allow DNA binding. After several washing steps the DNA was eluted from the column with sterile distilled water and stored at +4° C.

### 6.2 Plant DNA Isolation

Total plant DNA is isolated using a modified CTAB procedure (Murray & Thompson, 1980).

Plant leaves were collected and frozen in liquid nitrogen and ground to fine powder either using pestle and mortar or a Retsch mill. 500 μl of pre-warmed CTAB Buffer (65° C.) was added to 200 mg frozen sample material and incubated for 1 h at 65° C. in a re-circulating water bath, gently mixed by inverting from time to time. Samples were allowed to cool down for 5 minutes at RT before addition of 500 μl chloroform. The tubes were shacked for 30 minutes at RT and then centrifuged for 10 minutes at 10000 rpm at +4° C. The upper watery phase was transferred into a new tube and chilled Isopropanol was used to precipitate the DNA. After centrifugation for 10 min at 10.000 rpm at +4° C., the pellet was washed twice with 70% ethanol, centrifuged for 1 min at 10000 rpm at +4° C. and then air dried for 30 minutes. DNA was re-suspended in sterile distilled water and stored for later use at −20° C.

7. Cloning and E. coli Heat Shock Transformation
7.1 DNA Digestion with Restriction Enzymes

Restriction digests of plasmid DNA or total plant DNA with appropriate restriction endonucleases were performed in the corresponding buffer systems provided by the manufacturer at 37° C. overnight in a reaction volume of 30 μl. Restriction digests were heat inactivated by incubation at 65° C. for 20 minutes prior to any further utilization.

7.2 Ligation of Vector Backbone with DNA Fragments

The Rapid DNA Ligation Kit was used to ligate DNA fragments according to the protocol provided by the manufacturer and formula given below was used to determine the amount of insert based on a 3:1 molar ratio of insert to backbone DNA. The following reaction mixture was prepared in a reaction tube and incubated at 16° C. overnight (Table 9).

$$ng [ insert ] = \frac{3}{1} * ( \frac{size of insert in bp}{size of backbone in bp} ) * ng [ backbone ]$$

TABLE 9
Standard Ligation reaction set up.

| Substance | Amount |
|---|---|
| Linear backbone DNA | ~50 ng |
| Insert DNA | 3:1 molar ratio of insert to vector DNA |
| 10X ligation buffer | 1.5 μl |
| Ligase enzyme | 1.0 μl |
| H₂O | Add to 15.0 μl |

7.3 E. coli Transformation

All Plasmids (1 μl) and ligation products (5 μl) are transformed into chemically competent One Shot® E. coli by heat shock according to the manufacturer's protocol (Invitrogen™).

The cells were thawn on ice and after addition of the DNA they were incubated for 30 minutes on ice, followed by a heat shock treatment at 42° C. for 30 seconds and two minutes incubation on ice. 250 μl of SOC medium were added to 50 μl of initial cell culture and incubated at 37° C. for 1 h on shaker. 10 μl and 50 μl of the culture were plated on LB plates containing the suitable antibiotics for selection and grown at 37° C. overnight. The rest of the transformation culture is stored at +4° C. and if necessary, cells were pelleted, re-suspended in a smaller volume of liquid LB medium and plated out again.

Single colonies were picked and grown overnight in liquid LB-medium containing antibiotics, harvested and used for glycerol-stock and plasmid isolation.

### 7.4 Gene Synthesis

The nucleotide sequences encoding the transgenes (EDIII 1-4, EDIII 1) were codon usage optimized for the Lactuca sativa plastid and gene synthesis of these custom designed sequences was carried out by GeneArt (Germany).

### 7.5 Gateway® Cloning Procedure

All Gateway® cloning steps were carried out according to the protocol provided from Invitrogen™.

The BP-reaction is performed with the PstI linearized vector including the gene of interest (goi) and the Donor vector pDONR221™ and the BP enzyme mix at 25° C. for 1 h. The LR-reaction is performed with the previously produced Entry- and Destination-vectors and the LR enzyme mix for 1 h at 25° C. The resulting Entry vectors and Expression vectors are transformed into One Shot® OmniMAX™ E. coli cells by heat shock. The formula given below is used to calculate the correct amounts of goi and Donor vector with X for goi and Donor vector and N for the size in bp of the goi and the Donor vector, respectively. The standard set up for the carried out BP- and LR-reactions are given in Table 10. The BP- and the LR-reactions were stopped by adding 1 μL Proteinase K and incubation at 37° C. for 10 minutes.

$$ng [ X ] = ( 50 f mol ) * N * ( 660 [ \frac{fg}{f mol} ] ) * 1 ng / {10}^{6} fg$$

TABLE 10
Standard Set-up of BP- and LR-reaction.

| BP-reaction | 10 μL | | LR-reaction | 10 μL |
|---|---|---|---|---|
| goi | x | μL | | |
| pDONR221 ™ | 160 | ng | Destination vector | 160 ng |
| TE Buffer, pH 8.0 | 3 | μL | Entry vector | 120 ng |
| BP Clonase II | 2 | μL | LR Clonase II | 2 μL |

### 8. Vector Construction

8.1 Destination Vector pDEST-PN-L

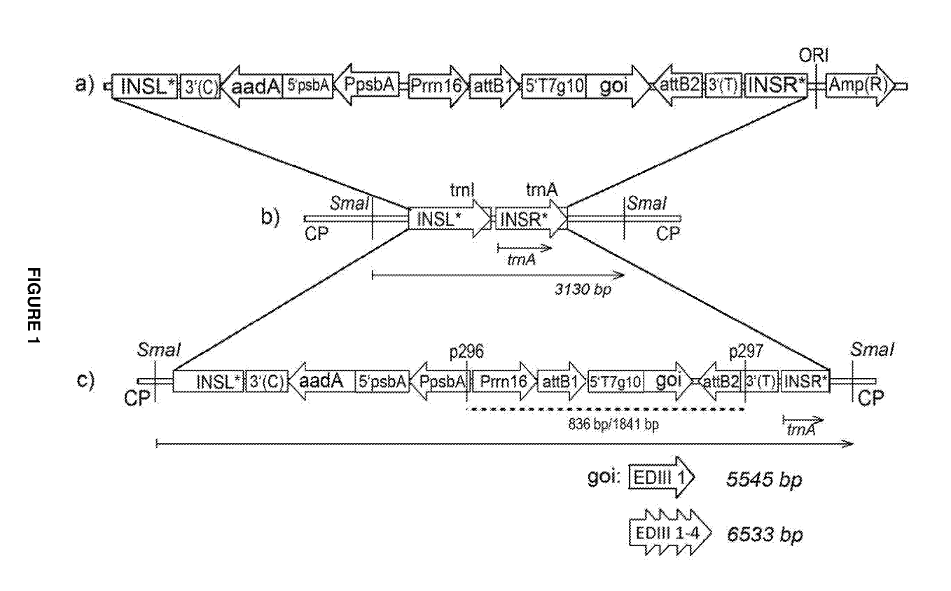

The lettuce specific plastid transformation vector pDEST-PN-L (FIG. 1 (e)) was obtained by insertion of the complete aadA expression cassette and the Gateway® cassette into a backbone vector containing the regions homologous to the trnI/trnA sequence from L. sativum (Ruhlmann et al. 2007).

Re-digest of the backbone vector pLettuce-MA (GeneArt, Germany) containing the lettuce specific sequences for homologous integration into the plastid genome with KpnI and SacII yielded the 3432 bp insert and the 4337 bp backbone which were separated by gel electrophoresis and the correct fragments were cut out and purified from the gel. The ligation product was transformed into ccdB Survival™ 2 T1R chemically competent E. coli and positive clones were selected on ampicillin and spectinomycin containing solid LB medium.

### 8.2 Entry Vectors and Expression Vectors

The entry vectors were created by the Gateway® BP Clonase® enzyme mix mediated transfer of the attB sites flanked gene of interest into the attP site bearing pDONR221™. The BP reactions were done according to the protocol provided by Invitrogen™. The vectors donating the attB site flanked gene of interest were linearized by PstI digest in order to maximize the recombination efficiency. The BP reaction products were transformed into OmniMax™ E. coli via heat shock, cells were plated on solid LB medium containing kanamycin and positive clones were identified by colony PCR with primers pM13F/. The gene of interest in the entry vectors was then transferred into the respective destination vector containing attR sites using the Invitrogen® LR Clonase® enzyme mix. The LR reactions were done according to the protocol provided by Invitrogen™. The LR reaction products were transformed into OmniMax™ E. coli via heat shock, cells were plated on solid LB medium containing ampicillin and spectinomycin and positive clones were identified by colony PCR with primers p296/p297. Correctness of all performed cloning steps was verified by sequencing of plasmid DNA isolated from PCR positive clones.

### 9. Plastid Transformation by Biolistic Bombardment Method

Transformation of chloroplasts and regeneration of transplastomic plants was achieved with the biolistic transformation method using a PDS-1000/He Particle Delivery System and following the modified protocol from (Verma et al, 2008). Table 11 lists the performed transformations and the generated transplastomic plant lines.

### 9.1 Preparation of Plant Material

Leaves of 6 weeks old plants grown under sterile conditions were harvested, placed on RMOP-medium facing the abaxial side up and incubated at 25° C. in the dark overnight.

### 9.2 Sterilization of Microcarrier

30 mg of gold particles were accurately weighed and transferred to 1.5 ml Eppendorf tube. 1 ml of 70% ethanol was added to the tube, vortexed for 15 minutes and centrifuged for 10 seconds at maximum speed. The supernatant was removed and the gold palette was washed again with 70% ethanol, washing was repeated for two more times. After third washing the supernatant was discarded and the gold particles were re-suspended in 500 μl of 50% glycerol with a final concentration of 60 mg/ml.

### 9.3 DNA Coating of Microcarrier

5 μl DNA (1 μg/μl), 50 μl 2.5 M CaCl2 and 20 μl 0.1 M spermidine were added while vortexing to 50 μl of sterile microcarriers re-suspended in glycerol. The mixture was incubated on ice for 10 minutes and then centrifuged for 1 min at 8 000 rpm. The supernatant was carefully removed and the pellet was first washed with 140 μl 70% ethanol and centrifuged 1 minute at 10000 rpm; second the pellet was washed with 140 μl of 100% ethanol for 1 minute and centrifuged at 10000 rpm. The supernatant was removed and the DNA-coated microcarriers were carefully re-suspended in 48 μl of 100% ethanol and kept on ice until used.

### 9.4 Bombardment

All the equipment and the bombardment chamber were sterilized with 70% ethanol. 6 μl of freshly prepared DNA coated gold particles were loaded on macro carries in macro carrier holder. The sterile rupture disk of 1100 psi was placed in the retaining cap and secured to the gas acceleration tube. Plant tissues were bombarded with DNA coated gold microcarriers in the vacuum chamber at a pressure of 1100 psi.

### 9.5 Selection and Regeneration of Transformed Plants

The bombarded leaf discs were placed on RMOP medium and incubated in the dark at 25° C. for two days. Then the leaves were cut into small pieces (˜5 mm2), transferred to RMOP medium containing spectinomycin and kept at 25° C. under standard light conditions. Three to four weeks after the transformation the resistant shoots started to regenerate and were transferred to fresh medium. In order to obtain homoplasmic plants transplastomic shoots were subjected to 2 additional rounds of regeneration on RMOP medium containing spectinomycin. Integration of the transgene expression cassette into the tobacco plastid genome was verified by a 2552 bp PCR product with primer p3/p4 PCR positive plantlets were used for further analysis. Presence of the transgene expression cassette in the lettuce plastid genome was verified by 836 bp PCR product for S16-PN-EDIII 1 and 1841 bp PCR fragment for S12-PN-EDIII 1-4 with primers p296/p297.

TABLE 11
Summary of performed transforamtion events

| Bombarded leaves | Transformation vector | Transplastomic plant lines |
|---|---|---|
| wtL | pEXP-PN-EDIII 1-L | S16-PN-EDIII 1 |
| wtL | pEXP-PN-EDIII 1-4-L | S12-PN-EDIII 1-4 |

### 10. Molecular Analysis to Verify the Transformants

### 10.1 Southern Blot Analysis

The Southern Blot analyses were carried out according to the protocol provided with the DIG-High Prime DNA Labeling and Detection Starter Kit II (Roche). Plant DNA was isolated from transplastomic and wild-type plants after three consecutive rounds of selection and subculture on spectinomycin containing RMOP medium and analyzed using DIG labeled probes (Table 12) that bind inside the transgene expression cassettes and the plastid genome. 10 μg of plant DNA was cut with SmaI (for S12-PN-EDIII 1-4 and S16-PN-EDIII 1), separated by electrophoresis in a 1 Agarose gel at 50 V overnight and transferred onto a positively charged nylon membrane by capillary action using the semi-dry transfer overnight. After immobilization of the DNA by baking the membrane at 80° C. for 2 h, the DNA was pre-hybridized for 3 h at 45° C. and hybridized with the specific labeled probe (Table 13) at 45° C. overnight to visualize the sequence of interest. Stringency washes were performed with 2×SSC+1 SDS at RT and 0.5×SSC+1 SDS at 65° C. After incubation in blocking solution for 30 minutes at RT and incubation in antibody solution for 30 minutes at RT, the membrane was washed twice with 1×WB, 1 ml of CSPD ready to use solution was applied to the membrane and incubated at 37° C. for 10 minutes. The signal was detected by exposure to X-ray film and developer and fixer solution were used to develop the X-ray film.

TABLE 12
Probes and their binding region.

| Probe | Size [bp] | Binding in |
|---|---|---|
| trnA | 665 | lettuce INSL region near trnA |

TABLE 13
Overview of plant samples and the respective probe
used for hybridization

| Plant line | Hybridization with probe |
|---|---|
| wtL | trnA |
| S12-PN-EDIII 1-4 | trnA |
| S16-PN-EDIII 1 | trnA |

### 10.2 WesternBlot Analysis

200 mg of frozen leave sample were ground into fine powder using liquid nitrogen and homogenized in 500 μl plant extraction buffer by vortexing for 3 minutes at RT. PEB II was used to extract total soluble protein (TSP) from all plant lines. The supernatant was collected after centrifugation for 10 minutes at 13000 rpm at +4°, aliquoted and stored at −20° C.

Alternatively, 200 mg of frozen leave sample were ground into fine powder using liquid nitrogen and homogenized in 500 μl PEB III by vortexing for 1 minute at RT in order to extract total protein (TP). 500 μl of Phenol were added to the plant cell extract, vortexed briefly and centrifuged at 13000 rpm for 10 minutes at +4° C. 200 μl of the upper green supernatant were transferred into a new tube and 1 ml of 0.1 M NH4OAc in Methanol was added and the proteins were precipitated for 3 h at −20° C. After centrifugation at 13000 rpm at +4° C. for 10 minutes the pellet was washed twice with 500 μl 0.1 M NH4OAc in Methanol and the air dried at RT. Finally the protein pellet was dissolved in 100 μl 1% SDS and stored at −20° C.

20 μl sample were mixed with 5 μl Laemmli Buffer, denatured at 95° C. for 10 minutes, spun down and loaded onto the 12% PAA gel. Proteins were separated by electrophoresis and then transferred onto the nitrocellulose membrane and blocked with 0.5% BSA in TBS-T for 1 h. The membrane was briefly rinsed with TBS-T and then incubated with the primary antibody 1:1000 diluted in TBS-T overnight at +4° C. The membrane was washed three times with TBS-T at RT and incubated for 1 h with alkaline phosphates conjugated goat anti-mouse IgG (Promega) as a secondary antibody diluted 1:10000 in TBS-T at RT. Proteins were detected by colorimetric reaction using either the AP color development Kit (Bio-Rad, USA) or with Sigmafast™ BCIP®/NBT (Sigma). Coomassie staining with Brilliant Blue G was carried out in order to verify equal loading amounts of proteins. The PAA gels were stained for 1 h at RT with the Coomassie staining solution and destained overnight in 10% Acetic acid.

### 10.3 Quantification of TSP and TP

Quantification of isolated TSP was done using the Bradford assay and following the instructions provided in the manufacturer's protocol. The BSA standards were prepared by diluting the provided 2 mg/ml Stock in PEB II, respectively (Table 14). The standards and samples were measured using the standard procedure where 1 ml ready to use Bradford Reagent is added to 20 μl sample, incubated at RT for 10 minutes and then measured at 595 nm. T standard curve was obtained as a regression equation and was used to calculate the concentration of TSP in the unknown samples. All measurements were carried out in technical duplicates. Quantification of TP was done using the BCA protein assay Kit (Pierce). The BSA standard dilution series was prepared by diluting the provided 2 mg/ml Stock in PEB III to the final concentrations given in Table 18. Assays were carried out according to the protocol provided with the Kit and the microplate procedure was uses where 25 μl of sample are mixed with 200 μl 1× Working reagent, incubated at 37° C. for 30 minutes and then measured at 652 nm. The standard curve was obtained by regression and the concentration of the unknown samples was calculated. All measurements were carried out in technical duplicates.

TABLE 14
BSA Standard dilution series

| Standard | Concentration [μg/ml] |
|---|---|
| 1 | 2000 |
| 2 | 1500 |
| 3 | 1000 |
| 4 | 750 |
| 5 | 500 |
| 6 | 250 |
| 7 | 125 |
| 8 | 25 |
| 9 | 0 |

### Results

### 11. Expression of Monovalent EDIII 1 and Tetravalent EDIII 1-4 Antigens in Lettuce Plastids

### 11.1 Vector Construction

The lettuce plastid transformation vector pDEST-PN-L was constructed by insertion of the aadA expression cassette and the Gateway® RfA between lettuce specific sites for homologous recombination. The vectors pEXP-PN-EDIII 1-L and pEXP-PN-EDIII 1-4-L (1(a)) used for lettuce plastid transformation were obtained by Gateway® cloning of the sequences for EDII 1 and EDIII 1-4 into the lettuce specific pDEST-PN-L. Homologous recombination into the intergenic spacer region between trnI and trnA in the IR region of the lettuce plastid genome (Figure a (b)) resulted in transplastomic plants carrying the corresponding transgene expression cassettes (FIG. 1 (c)).

### 11.2 Transplastomic Plant Regeneration and Characterization

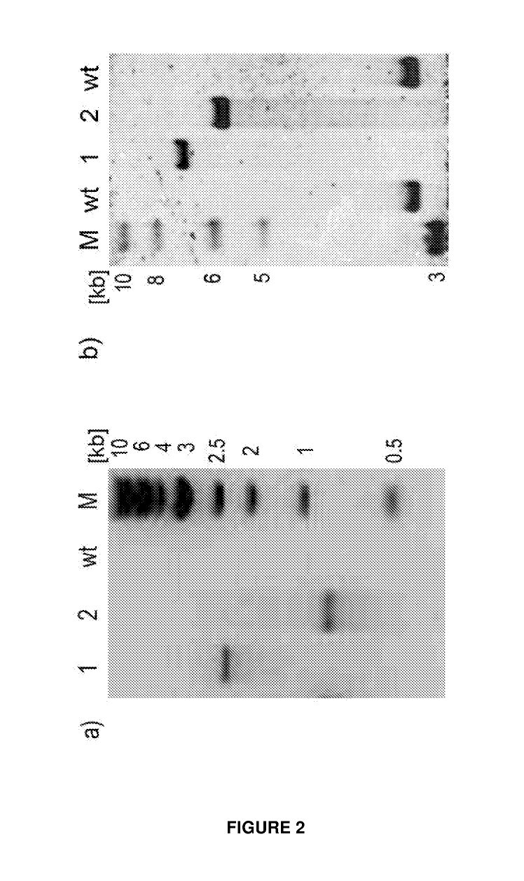

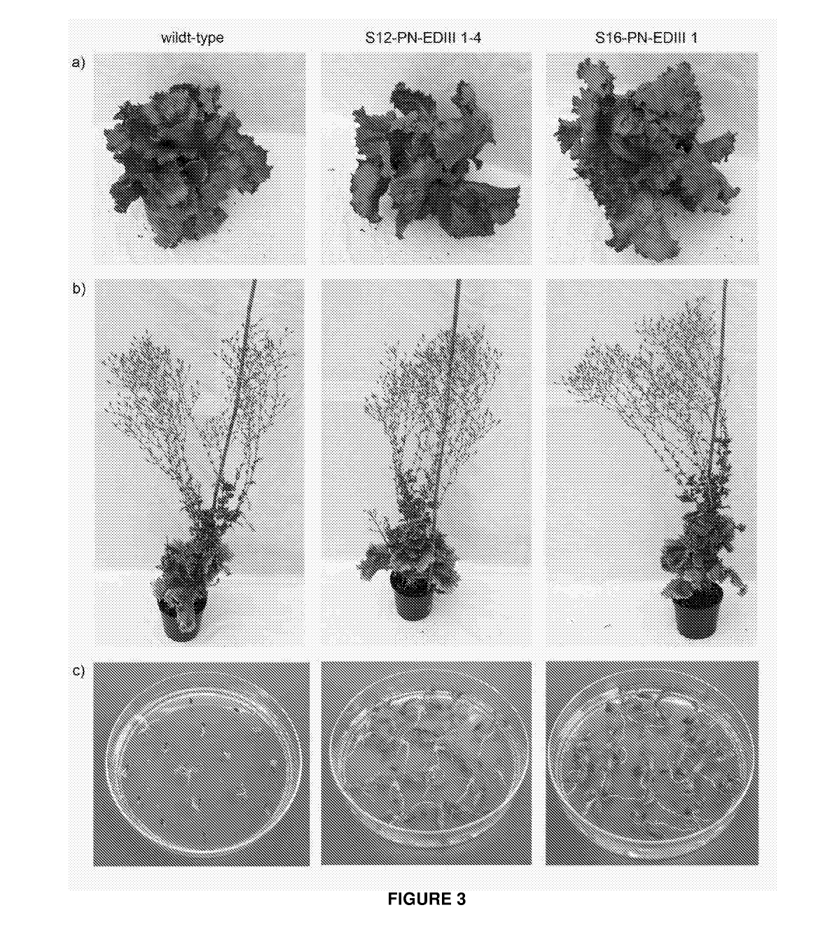

Transgenic shoots developing from callus tissue on RMOP medium containing spectinomycin were tested for transgene integration by PCR. Presence of the transgenic sequence in the plastid genome was shown by a PCR product of 1841 bp for EDIII 1-4 and 836 bp for EDIII 1 with primers p296/p297 (FIG. 2 (a)). The transplastomic plant lines (S12-PN-EDIII 1-4 and S16-PN-EDIII 1) were further characterized by Southern blot analysis. The homoplastomic state of both plant lines was verified by the presence of only the 5545 bp fragment (in S16-PN-EDIII 1) or the 6533 bp fragment (in S12-PN-EDIII 1-4) in transformed plants, compared to the 3130 bp fragment in wild-type (FIG. 2 (b)) after digest of total plant DNA with SmaI. No phenotypic alterations were visible on transplastomic plants growing in the greenhouse (FIG. 3 (a)) and flower set and seed development was normal. Plants were grown to full maturity (FIG. 3 (b)) and seeds harvested from transgenic plants were geminated on spectinomycin containing medium. The homogenous green phenotype of the seedlings proved the absence of segregation of the antibiotic resistance gene in the F1 generation (FIG. 3 (c)).

### 11.3 Analysis of the Expression of EDIII 1-4 and EDIII 1 Antigens

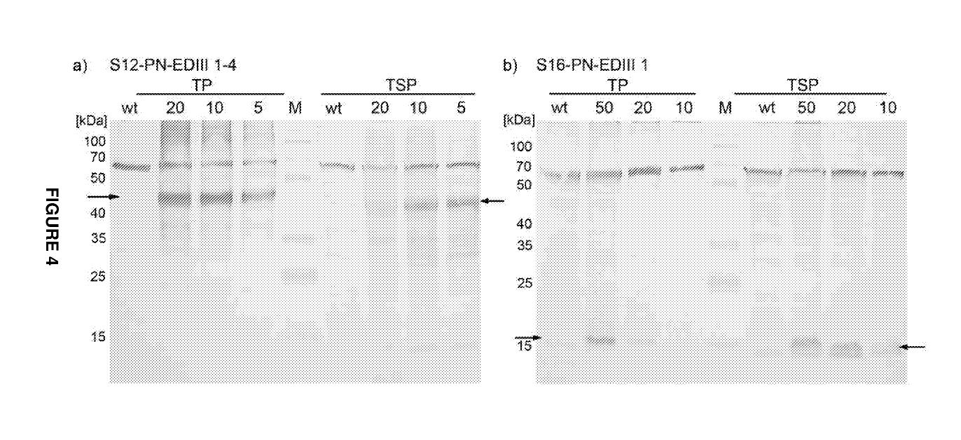

Total protein (TP) and total soluble protein (TSP) were isolated from plants growing in the greenhouse, quantified by BCA and Bradford assay and immunoblot analysis performed with an anti-dengue antibody detected both the 13 kDa EDIII 1 and the 47 kDa EDIII 1-4 in the respective plants (FIG. 4).

### LIST OF CERTAIN REFERENCES CITED IN THE DESCRIPTION

Allison S L, Schalich J, Stiasny K, Mandl C W, Heinz F X (2001) Mutational evidence for an internal fusion peptide in flavivirus envelope protein E. J Virol 75: 4268-4275
Batra G, Hapugoda M, Chaudhry S, al. e. (2007) Tetravalent dengue specific domain III based chimeric recombinant protein. Vol. WO2007034507.
Bhamarapravati N, Butrapet S, Chang J, al. e. (1996) Infectious dengue 2 virus PDK-53 as quadravalent vaccine.
Bhamarapravati N, Yoksan S. (2001) Attenuated strains of dengue virus and their use in a vaccine composition. Vol. EP1159968.
Brandler S, Lucas-Hourani M, Moris A, Frenkiel M-P, Combredet C, Février M, Bedouelle H, Schwartz O, Desprès P, Tangy F (2007) Pediatric Measles Vaccine Expressing a Dengue Antigen Induces Durable Serotype-specific Neutralizing Antibodies to Dengue Virus. PLoS NegI Trop Dis 1: e96
Brown D. (2004) Insertion of furin protease cleavage sites in membrane proteins and uses thereof.
Calisher C H, Karabatsos N, Dalrymple J M, Shope R E, Porterfield J S, Westaway E G, Brandt W E (1989) Antigenic relationships between flaviviruses as determined by cross-neutralization tests with polyclonal antisera. J Gen Virol 70 (Pt 1): 37-43
Chen Y, Maguire T, Hileman R E, Fromm J R, Esko J D, Linhardt R J, Marks R M (1997) Dengue virus infectivity depends on envelope protein binding to target cell heparan sulfate. Nat Med 3: 866-871
Chin J F, Chu J J, Ng M L (2007) The envelope glycoprotein domain III of dengue virus serotypes 1 and 2 inhibit virus entry. Microbes Infect 9: 1-6
Crill W D, Roehrig J T (2001) Monoclonal antibodies that bind to domain III of dengue virus E glycoprotein are the most efficient blockers of virus adsorption to Vero cells. J Virol 75: 7769-7773
Durbin A P, Karron R A, Sun W, Vaughn D W, Reynolds M J, Perreault J R, Thumar B, Men R, Lai C J, Elkins W R, Chanock R M, Murphy B R, Whitehead S S (2001) Attenuation and immunogenicity in humans of a live dengue virus type-4 vaccine candidate with a 30 nucleotide deletion in its 3′-untranslated region. Am J Trop Med Hyg 65: 405-413
Eckels K, Putnak J, Dubois D. (2000a) Multivalent dengue virus vaccine. Vol. WO0057907.
Eckels K, Putnak J, Dubois D, al. e. (2000b) Attenuated dengue-1 (-2, -3 and -4) virus vaccine. Vol. WO0057908 (WO0057909, WO0057904 and WO0057910).
Edelman R, Wasserman S S, Bodison S A, Putnak R J, Eckels K H, Tang D, Kanesa-Thasan N, Vaughn D W, Innis B L, Sun W (2003) Phase I trial of 16 formulations of a tetravalent live-attenuated dengue vaccine. Am J Trop Med Hyg 69: 48-60
Etemad B, Batra G, Raut R, Dahiya S, Khanam S, Swaminathan S, Khanna N (2008) An Envelope Domain III-based Chimeric Antigen Produced in Pichia pastoris Elicits Neutralizing Antibodies Against All Four Dengue Virus Serotypes. The American Journal of Tropical Medicine and Hygiene 79: 353-363
Frolov I, Frolova E, Mason P. (2007) Pseudoinfectious flavivirus and uses thereof.
Guirakhoo F, Arroyo J, Pugachev K V, Miller C, Zhang Z X, Weltzin R, Georgakopoulos K, Catalan J, Ocran S, Soike K, Ratterree M, Monath T P (2001) Construction, safety, and immunogenicity in nonhuman primates of a chimeric yellow fever-dengue virus tetravalent vaccine. J Virol 75: 7290-7304
Guzman M, Vázquez S, Kouri G. (2009) Dengue: where are we today? Malays J Med Sci., Vol. 16 (3), pp. 4-11.
Guzman M G, Hermida L, Bernardo L, Ramirez R, Guillen G (2010) Domain III of the envelope protein as a dengue vaccine target. Expert Rev Vaccines 9: 137-147
Halstead S B (1988) Pathogenesis of dengue: challenges to molecular biology. Science 239: 476-481
Halstead S B, Marchette N J (2003) Biologic properties of dengue viruses following serial passage in primary dog kidney cells: studies at the University of Hawaii. Am J Trop Med Hyg 69: 5-11
Hombach J, Barrett A D, Cardosa M J, Deubel V, Guzman M, Kurane I, Roehrig J T, Sabchareon A, Kieny M P (2005) Review on flavivirus vaccine development. Proceedings of a meeting jointly organised by the World Health Organization and the Thai Ministry of Public Health, 26-27 Apr. 2004, Bangkok, Thailand. Vaccine 23: 2689-2695
Ivy J, Nakano E, Clements D. (2000) Subunit immunogenic composition against dengue infection.
Kanesa-thasan N, Sun W, Kim-Ahn G, Van Albert S, Putnak J R, King A, Raengsakulsrach B, Christ-Schmidt H, Gilson K, Zahradnik J M, Vaughn D W, Innis B L, Saluzzo J F, Hoke C H, Jr. (2001) Safety and immunogenicity of attenuated dengue virus vaccines (Aventis Pasteur) in human volunteers. Vaccine 19: 3179-3188
Martinez C A, Topal E, Giulietti A M, Talou J R, Mason H (2010) Exploring different strategies to express Dengue virus envelope protein in a plant system. Biotechnol Lett 32: 867-875
McDonald W, Powell T, Price A, Becker R. (2009) Compositions of dengue viral proteins and methods of use.
Modis Y, Ogata S, Clements D, Harrison S C (2003) A ligand-binding pocket in the dengue virus envelope glycoprotein. Proceedings of the National Academy of Sciences 100: 6986-6991
Monath T (1997) Yellow fever in vaccines. Plotkin S, Orenstein W (eds), 3rd edition edn, pp 815-880. Philadelphia: W B Saunders Co.
Morrison D, Legg T J, Billings C W, Forrat R, Yoksan S, Lang J (2010) A novel tetravalent dengue vaccine is well tolerated and immunogenic against all 4 serotypes in flavivirus-naive adults. J Infect Dis 201: 370-377
Pasteur S. (2011) Study of a Novel Tetravalent Dengue Vaccine in Healthy Children and Adolescents Aged 9 to 16 Years in Latin America. NCT01374516. Clinical Trials database of the US NIH.
Ruhlman T, Ahangari R, Devine A, Samsam M, Daniell H (2007) Expression of cholera toxin B-proinsulin fusion protein in lettuce and tobacco chloroplasts—oral administration protects against development of insulitis in non-obese diabetic mice. Plant Biotechnology Journal 5: 495-510
Sabchareon A, Wallace D, Sirivichayakul C, Limkittikul K, Chanthavanich P, Suvannadabba S, Jiwariyavej V, Dulyachai W, Pengsaa K, Wartel T A, Moureau A, Saville M, Bouckenooghe A, Viviani S, Tornieporth N G, Lang J (2012) Protective efficacy of the recombinant, live-attenuated, CYD tetravalent dengue vaccine in Thai schoolchildren: a randomised, controlled phase 2b trial. The Lancet 380: 1559-1567
Staub J M, Maliga P (1993) Accumulation of D1 polypeptide in tobacco plastids is regulated via the untranslated region of the psbA mRNA. EMBO J 12(2): 601-606
Sun W, Edelman R, Kanesa-Thasan N, Eckels K H, Putnak J R, King A D, Houng H S, Tang D, Scherer J M, Hoke C H, Jr., Innis B L (2003) Vaccination of human volunteers with monovalent and tetravalent live-attenuated dengue vaccine candidates. Am J Trop Med Hyg 69: 24-31
Swaminathan S, Batra G, Khanna N (2010) Dengue vaccines: state of the art. Expert Opinion on Therapeutic Patents 20: 819-835
Verma D, Samson N P, Koya V, Daniell H (2008) A protocol for expression of foreign genes in chloroplasts. Nat Protoc 3(4): 739-758
Ye G N, Hajdukiewicz P T, Broyles D, Rodriguez D, Xu C W, Nehra N, Staub J M (2001) Plastid-expressed 5-enolpyruvylshikimate-3-phosphate synthase genes provide high level glyphosate tolerance in tobacco. Plant J 25(3): 261-270

### Embodiments of the Invention

1. A recombinant DNA molecule comprising a promoter that is functional in a plant cell to cause the production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

2. The recombinant DNA molecule according to item 2, wherein EDIII-1 comprises the amino acid sequence set forth in SEQ ID NO: 1, EDIII-2 comprises the amino acid sequence set forth in SEQ ID NO: 2, EDIII-3 comprises the amino acid sequence set forth in SEQ ID NO: 3, and EDIII-4 comprises the amino acid sequence set forth in SEQ ID NO: 4.

3. The recombinant DNA molecule according to item 1 or 2, wherein the variant of EDIII-1 comprises an amino acid sequence which has at least about 80% sequence identity to the amino acid sequence set forth in SEQ ID NO: 1, the variant of EDIII-2 comprises an amino acid sequence which has at least about 80% sequence identity to the amino acid sequence set forth in SEQ ID NO: 2, the variant of EDIII-3 comprises an amino acid sequence which has at least about 80% sequence identity to the amino acid sequence set forth in SEQ ID NO: 3, and the variant of EDIII-4 comprises an amino acid sequence which has at least about 80% sequence identity to the amino acid sequence set forth in SEQ ID NO: 4.

4. The recombinant DNA molecule according to any one of items 1 to 3, wherein adjacent EDIIIs are joined by linkers.

5. The recombinant DNA molecule according to item 4, wherein the linkers are peptide linkers, such as polyglycine linkers.

6. The recombinant DNA molecule according to item 5, wherein said linkers are composed of from about 1 to about 20 amino acids, such as from about 2 to about 10 amino acids, such as from about 3 to about 7 amino acids, such as about 5 amino acids.

7. The recombinant DNA molecule according to any one of item 1 to 6, wherein the polyprotein comprises the amino acid sequence set forth in SEQ ID NO: 5 or an amino acid sequence having at least about 80% sequence identify to the amino acid sequence set forth in SEQ ID NO: 5.

8. The recombinant DNA molecule according to any one of items 1 to 7, wherein the promoter is a promoter functional in a Lactuca sativa cell.

9. The recombinant DNA molecule according to any one of items 1 to 8, wherein the promoter is selected from the group consisting of Lactuca sative psbA promoter, tabacco psbA promoter, tobacco rrn16 PEP+NEP promoter, CaMV 35S promoter, 19S promoter, tomate E8 promoter, nos promoter, Mac promoter, pet E promoter and ACT1 promoter.

10. The recombinant DNA molecule according to any one of items 1 to 9, wherein the promoter is the Lactuca sative psbA promoter or tabacco psbA promoter.

11. The recombinant DNA molecule according to any one of items 1 to 10, further comprising at least one regulatory element selected from the group consisting of a 5′ untranslated region (5′ UTR), 3′ untranslated region (3′ UTR), and transit peptide region.

12. The recombinant DNA molecule according to any one of items 1 to 11, further comprising a 5′ untranslated region selected from the group consisting of the 5′ untranslated region of the Lactuca sative psbA gene or the 5′ untranslated region of the tabacco psbA gene.

13. The recombinant DNA molecule according to any one of items 1 to 11, further comprising a 3′ untranslated region selected from the group consisting of the 3′ untranslated region of the Lactuca sative psbA gene, the tabacco psbA gene or the tabacco rbcL gene.

14. A vector comprising the recombinant DNA molecule according to any one of items 1 to 13.

15. The vector according to item 14, wherein the vector is an expression vector.

16. The vector according to item 14, wherein the vector is a transformation vector.

17. The vector according to item 16, wherein the vector is plastid transformation vector for stably transforming a plastid.

18. The vector according to any one of items 14 to 17, further comprising a first flanking DNA sequence and second flanking DNA sequence each of which is homologous to sequences in a spacer region of the genome of said plastid.

19. The vector according to item 18, wherein said spacer region is a transcriptionally active spacer region.

20. The vector according to item 18, wherein said spacer region is the spacer region between the trnI and trnA coding sequences in the plastid genome.

21. The vector according to any one of items 18 to 20, wherein the first flanking DNA sequence is homologous to the trnI coding sequence and the second flanking DNA sequence is homologous to the trnA coding sequence in the plastid genome.

22. The vector according to any one of items 14 to 21, wherein said vector comprises, as operably linked components arranged in the 5′ to 3′ direction, said first flanking DNA sequence, said promoter, said 5′ untranslated region, said nucleotide sequence encoding said polyprotein, said 3′ untranslated region, and said second flanking DNA sequence.

23. A transgenic plastid comprising a recombinant DNA molecule according to any one of items 1 to 13.

24. The plastid according to item 23, wherein the plastid is selected from the group consisting of chloroplast, chromoplast, gerentoplast, etioplast, leucoplast.

25. The plastid according to item 24, wherein the leucoplast is an amyloplast, proteinoplast or elaioplast.

26. The plastid according to any one of items 23 to 25, wherein said plastid is derived from a plant or plant cell of the Asteraceae family.

27. The plastid according to any one of items 23 to 26, wherein said plastid is derived from a plant or plant cell of the Lactuca genus.

28. The plastid according to any one of items 23 to 27, wherein said plastid is derived from a Lactuca sativa plant or cell.

29. A transgenic plant cell comprising a recombinant DNA molecule according to any one of items 1 to 13.

30. The transgenic plant cell according to item 29, wherein said recombinant DNA molecule is stably integrated in a plant cell genome.

31. The transgenic plant cell according to item 29, wherein said recombinant DNA molecule is stably integrated into the genome of a plant cell plastid.

32. The transgenic plant cell according to item 29, wherein said recombinant DNA molecule is stably integrated into a chromosome in a plant cell nucleus.

33. The transgenic plant cell according to any one of items 29 to 32, wherein said plant cell is part of a transgenic plant.

34. The transgenic plant cell according to any one of items 29 to 32, wherein said plant cell is in a plant seed.

35. The transgenic plant cell according to any one of items 29 to 34, wherein said plant cell is a cell of the Asteraceae family.

36. The transgenic plant cell according to any one of items 29 to 35, wherein said plant cell is a cell of the Lactuca genus.

37. The transgenic plant cell according to any one of items 29 to 36, wherein said plant cell is a Lactuca sativa cell.

38. A transgenic plant cell comprising the plastid according to any one of items 23 to 28.

39. A transgenic plant or transgenic part of said plant comprising a recombinant DNA molecule according to any one of items 1 to 13.

40. The transgenic plant or transgenic part of said plant according to item 39, wherein said recombinant DNA molecule is stably integrated in a plant cell genome.

41. The transgenic plant or transgenic part of said plant according to item 39, wherein said recombinant DNA molecule is stably integrated into the genome of a plant cell plastid.

42. The transgenic plant or transgenic part of said plant according to item 39, wherein said recombinant DNA molecule is stably integrated into a chromosome in a plant cell nucleus.

43. A transgenic plant or transgenic part of said plant comprising a plurality of plant cells according to any one of items 29 to 38.

44. The transgenic plant or transgenic part of said plant according to any one of items 39 to 43, which is homogenous for said recombinant DNA molecule.

45. The transgenic plant or transgenic part of said plant according to any one of items 39 to 44, wherein said plant is a plant of the Asteraceae family.

46. The transgenic plant or transgenic part of said plant according to any one of items 39 to 45, wherein said plant is a plant of the Lactuca genus.

47. The transgenic plant or transgenic part of said plant according to any one of items 39 to 46, wherein said plant is a Lactuca sativa plant.

48. The transgenic plant cell, transgenic plant or transgenic part of said plant according to any one of item 29 to 47 for use in vaccinating a subject against dengue virus.

49. A transgenic seed comprising a recombinant DNA molecule according to any one of items 1 to 13.

50. A transgenic seed comprising a plurality of plant cells according to any one of items 29 to 38.

51. The transgenic seed according to claim 49 or 50, wherein said seed is derived from the transgenic plant according to any one of items 39 to 47.

52. A method of producing a transgenic plastid comprising the step of:

Introducing a recombinant DNA molecule according to any one of items 1 to 13 or a vector according to any one of claims 14 to 21 into a plastid.

53. The method according to item 52, wherein the plastid is selected from the group consisting of chloroplast, chromoplast, gerentoplast, etioplast, leucoplast.

54. The method according to item 52 or 53, further comprising the step of cultivating said transgenic plastid.

55. A method of producing a transgenic plant cell, a transgenic plant or a transgenic plant part comprising the step of:

Introducing a recombinant DNA molecule according to any one of items 1 to 13 or a vector according to any one of items 14 to 21 into a plant cell, a plant or plant part.

56. The method according to item 55, further comprising the step of cultivating said transgenic plant cell, transgenic plant or transgenic plant part.

57. The method according to item 55 or 56, further comprising regenerating a transgenic plant from said transgenic plant cell.

58. The method according to any one of items 55 to 57, further comprising harvesting seeds of said transgenic plant.

59. Method of producing a transgenic plant comprising the steps of:

(a) planting a transgenic seed according to any one of items 49 to 51; and
(b) growing a plant from said seed.

60. A method of producing a tetravalent chimeric dengue virus antigen comprising the steps of:

Producing a transgenic plant comprising a recombinant DNA molecule according to any one of items 1 to 13; and
Incubating said plant under conditions wherein said plant expresses said antigen.

61. A vaccine comprising a transgenic plant cell, a transgenic plant or a transgenic part of said plant according to any one of items 29 to 47 optionally together with one or more pharmaceutically acceptable excipients.

62. The vaccine according to item 61, wherein the transgenic plant or transgenic part of said plant is formulated into said vaccine in a raw or processed form.

63. The vaccine according to item 61 or 62, wherein said vaccine is capable of eliciting an immune response upon administration to a subject.

64. The vaccine according to any one of items 61 to 63, wherein said vaccine is orally administrable.

65. The vaccine according to any one of items 61 to 64 for use in the vaccination of subject against dengue virus.

66. The vaccine according to item 65, wherein the subject is a mammal.

67. The vaccine according to item 65 or 66, wherein the subject is a human.

68. A method of producing a vaccine comprising the steps of:

a) Producing a transgenic plant cell, a transgenic plant or a transgenic part of said plant comprising a recombinant DNA molecule according to any one of items 1 to 13;
b) incubating said transgenic plant cell, said transgenic plant or said transgenic part of said plant under conditions wherein said plant cell, plant or part of said plant expresses the polyprotein encoded by said recombinant DNA molecule;
c) harvesting said transgenic plant cell, said transgenic plant or said transgenic part of said plant; and
d) formulating said transgenic plant cell, said transgenic plant or said transgenic part of said plant into a vaccine, optionally together with one or more pharmaceutically acceptable excipients.

69. The method according to item 68, wherein said transgenic plant cell, said transgenic plant or said transgenic part of said plant is formulated in its raw form.

70. The method according to item 68, wherein said transgenic plant cell, said transgenic plant or said transgenic part of said plant is formulated in a processed form.

71. The method according to item 70, wherein the processed form is an extract of said transgenic plant or transgenic part of said plant.

72. The method according to any one of items 68 to 71, wherein said vaccine is capable of eliciting an immune response upon administration to a subject.

73. A method of vaccinating a subject against dengue virus, the method comprising the step of:

administering an effective amount of the vaccine according to any one of items 61 to 67 to a subject.

74. Use of a transgenic plant cell, a transgenic plant or a transgenic part of said plant according to any one of items 29 to 47 in the manufacture of a medicinal product for vaccination of a subject against dengue virus.

75. An isolated nucleic acid molecule comprising the nucleotide sequences set forth in SEQ ID NOS: 7, 8, 9 and 10, or nucleotide sequences having at least about 80% sequence identity thereto.

1. A transgenic plastid derived from a plant cell of the Asteraceae family comprising a recombinant DNA molecule comprising a promoter that is functional in a plant cell of the Asteraceae family, which induces production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

2-23. (canceled)

24. The transgenic plastid according to claim 1, wherein EDIII-1 comprises the amino acid sequence set forth in SEQ ID NO: 1, EDIII-2 comprises the amino acid sequence set forth in SEQ ID NO: 2, EDIII-3 comprises the amino acid sequence set forth in SEQ ID NO: 3, and EDIII-4 comprises the amino acid sequence set forth in SEQ ID NO: 4.

25. The plastid according to claim 1, wherein the plastid is a chloroplast.

26. The plastid according to claim 1, wherein said plastid is derived from a Lactuca sativa plant or cell.

27. A transgenic plant cell of the Asteraceae family comprising a recombinant DNA molecule comprising a promoter that is functional in a plant cell of the Asteraceae family. which induces production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDIII-4) or a variant thereof.

28. The transgenic plant cell of the Asteraceae family according to claim 27, wherein EDIII-1 comprises the amino acid sequence set forth in SEQ ID NO: 1, EDIII-2 comprises the amino acid sequence set forth in SEQ ID NO: 2, EDIII comprises the amino acid sequence set forth in SEQ ID NO: 3, and EDI 11-4 comprises the amino acid sequence set forth in SEQ ID NO: 4.

29. The transgenic plant cell according to claim 27, wherein said recombinant DNA molecule is stably integrated in a plant cell genome.

30. The transgenic plant cell according to claim 29, wherein said recombinant DNA molecule is stably integrated into the genome of a plant cell plastid.

31. The transgenic plant cell according claim 27, wherein said plant cell is a Lactuca sativa cell.

32. A transgenic plant of the Asteraceae family or transgenic part of said plant comprising a recombinant DNA molecule comprising a promoter that is functional in a plant cell of the Asteraceae family, which induces production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDI 11-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDI 11-4) or a variant thereof.

33. The transgenic plant or transgenic part of said plant according to claim 32, wherein EDIII-1 comprises the amino acid sequence set forth in SEQ ID NO: 1, EDIII-2 comprises the amino acid sequence set forth in SEQ ID NO: 2, EDIII-3 comprises the amino acid sequence set forth in SEQ ID NO: 3, and EDIII-4 comprises the amino acid sequence set forth in SEQ ID NO: 4.

34. The transgenic plant or transgenic part of said plant according to claim 32, wherein said recombinant DNA molecule is stably integrated in a plant cell genome.

35. The transgenic plant or transgenic part of said plant according to claim 32, wherein said recombinant DNA molecule is stably integrated into the genome of a plant cell plastid.

36. The transgenic plant or transgenic part of said plant according to claim 32, wherein said plant is a Lactuca sativa plant.

37. A method for inducing an immunogenic response in a subject comprising:
providing the transgenic plant cell of claim 27, or a transgenic plant or a part thereof comprising said transgenic plant cell to a subject in need of an immunogenic response to Dengue virus.

38. The method according to claim 37, wherein the subject is a human.

39. A transgenic seed derived from a plant or plant cell of the Asteraceae family comprising a recombinant DNA molecule comprising a promoter that is functional in a plant cell of the Asteraceae family, which induces production of an mRNA molecule and that is operably linked to a nucleotide sequence that encodes a polyprotein comprising the immunoglobulin-like domain of the envelope (E) protein of the dengue (DEN) virus serotype 1 (EDIII-1) or a variant thereof, the immunoglobulin-like domain of the envelope (E) protein of the dengue (DEN) virus serotype 2 (EDIII-2) or a variant thereof, the immunoglobulin-like domain of the envelope (E) protein of the dengue (DEN) virus serotype 3 (EDIII-3) or a variant thereof, and the immunoglobulin-like domain of the envelope (E) protein of the dengue (DEN) virus serotype 4 (EDI 11-4) or a variant thereof.

40. The transgenic seed according to claim 39, wherein EDIII-1 comprises the amino acid sequence set forth in SEQ ID NO: 1, EDIII-2 comprises the amino acid sequence set forth in SEQ ID NO: 2, EDIII-3 comprises the amino acid sequence set forth in SEQ ID NO: 3, and EDIII-4 comprises the amino acid sequence set forth in SEQ ID NO: 4.

41. The transgenic seed according to claim 39, wherein said seed is derived from a Lactuca sativa plant.

42. An immunogenic composition comprising a transgenic plant cell according to claim 27, or a transgenic plant or part thereof comprising said transgenic plant cell, optionally together with one or more pharmaceutically acceptable excipients.

43. The immunogenic composition according to claim 42, wherein said immunogenic composition is orally administrable.

44. The immunogenic composition according to claim 43, wherein the subject is a human.


SEQUENCE LISTING (35 sequences)

SEQ ID NO: 1 - protein, 116 aa, Dengue virus
MSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKGGGGGENLYFQG

SEQ ID NO: 2 - protein, 116 aa, Dengue virus
MSYSMCTGKFKVVKEIAETQHGTIVIRVQYEGDGSPCKTPFEIMDLEKRHVLGRLTTVNPIVTEKDSPVNIEAEPPFGDSYIIIGVEPGQLKLDWFKKGSSIGQGGGGGENLYFQG

SEQ ID NO: 3 - protein, 116 aa, Dengue virus
MSYAMCLNTFVLKKEVSETQHGTILIKVEYKGEDAPCKIPFSTEDGQGKAHNGRLITANPVVTKKEEPVNIEAEPPFGESNIVIGIGDKALKINWYRKGSSIGKGGGGGENLYFQG

SEQ ID NO: 4 - protein, 115 aa, Dengue virus
MSYTMCSGKFSIDKEMAETQHGTTVVKVKYEGAGAPCKVPIEIRDVNKEKVVGRIISPTPFAENTNSVTNIELERPLDSYIVIGVGDSALTLHWFRKGSSIGKGGGGGENLYFQG

SEQ ID NO: 5 - protein, 463 aa, Artificial Sequence
  chimeric tetravalent dengue virus antigen
MSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKGGGGGENLYFQGMSYSMCTGKFKVVKEIAETQHGTIVIRVQYEGDGSPCKTPFEIMDLEKRHVLGRLTTVNPIVTEKDSPVNIEAEPPFGDSYIIIGVEPGQLKLDWFKKGSSIGQGGGGGENLYFQGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYRKGSSIGKGGGGGENLYFQGMSYTMCSGKFSIDKEMAETQHGTTVVKVKYEGAGAPCKVPIEIRDVNKEKVVGRIISPTPFAENTNSVTNIELERPLDSYIVIGVGDSALTLHWFRKGSSIGKGGGGGENLYFQG

SEQ ID NO: 6 - DNA, 1392 bp, Artificial Sequence
  nucleotide sequence coding for chimeric tetravalent dengue virus antigen
atgtcttatgtaatgtgtactggttcttttaaattagaaaaagaagtagctgaaactcaacatggaactgtattagtacaagtaaaatatgaaggtactgatgctccttgtaaaattcctttttcttctcaagatgaaaaaggtgtaactcaaaatggaagacttattactgctaatcctattgttactgataaagaaaaacctgtaaatattgaagctgaacctccttttggtgaatcttatattgttgtaggtgctggtgaaaaagcattaaaattatcttggtttaaaaaaggttcttctattggaaaaggaggtggtggaggagaaaatttatattttcaaggtatgtcttattctatgtgtactggaaaatttaaagttgtaaaagaaattgctgaaactcaacatggaactattgtaattcgagttcaatatgaaggagatggttctccttgtaaaactccttttgaaattatggatttagaaaaaagacatgttttaggacgattaactactgtaaatcctattgttactgaaaaagattctcctgtaaatattgaagctgaacctccttttggtgattcttatattattattggtgtagaacctggacaattaaaattagattggtttaaaaaaggatcttctattggtcaaggaggtggtggaggagaaaatttatattttcaaggtatgtcttatgctatgtgtttaaatacttttgttttaaaaaaagaagtttctgaaactcaacatggaactattcttattaaagttgaatataaaggtgaagatgctccttgtaaaattcctttttctactgaagatggacaaggaaaagctcataatggaagattaataactgctaatcctgttgtaactaaaaaagaagaacctgttaatattgaagctgaacctccttttggtgaatctaatattgtaattggtattggagataaagcattaaaaataaattggtatagaaaaggttcttctattggtaaaggaggtggtggaggagaaaatttatattttcaaggtatgtcttatactatgtgttctggaaaattttctattgataaagaaatggctgaaactcaacatggaactactgtagttaaagtaaaatatgaaggtgctggagcaccttgtaaagtacctattgaaattcgagatgttaataaagaaaaagtagtaggaagaattatttctcctactccttttgctgaaaatactaattctgtaactaatattgaattagaaagacctttagattcttatattgtaattggagtaggagattctgctttaactttacattggtttagaaaaggatcttctattggaaaaggaggtggtggaggagaaaatttatattttcaaggttaa

SEQ ID NO: 7 - DNA, 348 bp, Artificial Sequence
  codon optimized sequence (EDIII-1)
atgtcttatgtaatgtgtactggttcttttaaattagaaaaagaagtagctgaaactcaacatggaactgtattagtacaagtaaaatatgaaggtactgatgctccttgtaaaattcctttttcttctcaagatgaaaaaggtgtaactcaaaatggaagacttattactgctaatcctattgttactgataaagaaaaacctgtaaatattgaagctgaacctccttttggtgaatcttatattgttgtaggtgctggtgaaaaagcattaaaattatcttggtttaaaaaaggttcttctattggaaaaggaggtggtggaggagaaaatttatattttcaaggt

SEQ ID NO: 8 - DNA, 348 bp, Artificial Sequence
  codon optimized sequence (EDIII-2)
atgtcttattctatgtgtactggaaaatttaaagttgtaaaagaaattgctgaaactcaacatggaactattgtaattcgagttcaatatgaaggagatggttctccttgtaaaactccttttgaaattatggatttagaaaaaagacatgttttaggacgattaactactgtaaatcctattgttactgaaaaagattctcctgtaaatattgaagctgaacctccttttggtgattcttatattattattggtgtagaacctggacaattaaaattagattggtttaaaaaaggatcttctattggtcaaggaggtggtggaggagaaaatttatattttcaaggt

SEQ ID NO: 9 - DNA, 348 bp, Artificial Sequence
  codon optimized sequence (EDIII-3)
atgtcttatgctatgtgtttaaatacttttgttttaaaaaaagaagtttctgaaactcaacatggaactattcttattaaagttgaatataaaggtgaagatgctccttgtaaaattcctttttctactgaagatggacaaggaaaagctcataatggaagattaataactgctaatcctgttgtaactaaaaaagaagaacctgttaatattgaagctgaacctccttttggtgaatctaatattgtaattggtattggagataaagcattaaaaataaattggtatagaaaaggttcttctattggtaaaggaggtggtggaggagaaaatttatattttcaaggt

SEQ ID NO: 10 - DNA, 345 bp, Artificial Sequence
  codon optimized sequence (EDIII-4)
atgtcttatactatgtgttctggaaaattttctattgataaagaaatggctgaaactcaacatggaactactgtagttaaagtaaaatatgaaggtgctggagcaccttgtaaagtacctattgaaattcgagatgttaataaagaaaaagtagtaggaagaattatttctcctactccttttgctgaaaatactaattctgtaactaatattgaattagaaagacctttagattcttatattgtaattggagtaggagattctgctttaactttacattggtttagaaaaggatcttctattggaaaaggaggtggtggaggagaaaatttatattttcaaggt

SEQ ID NO: 11 - protein, 15 aa, Dengue virus
KFKVVKEIAETQHGT

SEQ ID NO: 12 - DNA, 20 bp, Artificial Sequence
  Oligonucleotide Primer
acccatggcttctaaaggag

SEQ ID NO: 13 - DNA, 20 bp, Artificial Sequence
  Oligonucleotide Primer
agacagcgacgggttctctg

SEQ ID NO: 14 - DNA, 20 bp, Artificial Sequence
  Oligonucleotide Primer
gatccgagccatagaatttc

SEQ ID NO: 15 - DNA, 21 bp, Artificial Sequence
  Oligonucleotide Primer
tgctggccgtacatttgtacg

SEQ ID NO: 16 - DNA, 20 bp, Artificial Sequence
  Oligonucleotide Primer
tacccgggaattgtgacctc

SEQ ID NO: 17 - DNA, 20 bp, Artificial Sequence
  Oligonucletide Primer
agagtccgaccacaacgacc

SEQ ID NO: 18 - DNA, 22 bp, Artificial Sequence
  Oligonucletide Primer
gctgaaactcaacatggaactg

SEQ ID NO: 19 - DNA, 20 bp, Artificial Sequence
  Oligonucleotide Primer
atgctttttcaccagcacct

SEQ ID NO: 20 - DNA, 20 bp, Artificial Sequence
  Oligonucleotide Primer
ttgctgaaactcaacatgga

SEQ ID NO: 21 - DNA, 20 bp, Artificial Sequence
  Oligonucleotide Primer
ccaaaaggaggttcagcttc

SEQ ID NO: 22 - DNA, 21 bp, Artificial Sequence
  Oligonucleotide primer
tgaagatggacaaggaaaagc

SEQ ID NO: 23 - DNA, 20 bp, Artificial Sequence
  Oligonucleotide Primer
ctccaccacctcctttacca

SEQ ID NO: 24 - DNA, 24 bp, Artificial Sequence
  Oligonucleotide Primer
actactcaagctgcattatatacc

SEQ ID NO: 25 - DNA, 22 bp, Artificial Sequence
  Oligonucleotide Primer
gcaccttttactaagatcaatg

SEQ ID NO: 26 - DNA, 20 bp, Artificial Sequence
  Oligonucleotide Primer
ggaggtaggatgggcagttg

SEQ ID NO: 27 - DNA, 20 bp, Artificial Sequence
  Oligonucleotide Primer
ggactcgaaccgctgacatc

SEQ ID NO: 28 - DNA, 20 bp, Artificial Sequence
  Oligonucleotide Primer
ggactcgaaccgctgacatc

SEQ ID NO: 29 - DNA, 20 bp, Artificial Sequence
  Oligonucleotide Primer
aacgaccttttggaaacttc

SEQ ID NO: 30 - DNA, 20 bp, Artificial Sequence
  Oligonucleotide Primer
tctgtgagcgtgacggtggt

SEQ ID NO: 31 - DNA, 20 bp, Artificial Sequence
  Oligonucleotide Primer
ttacgcgaacgcgaagtccg

SEQ ID NO: 32 - DNA, 16 bp, Artificial Sequence
  Oligonucleotide Primer
gtaaaacgacggccag

SEQ ID NO: 33 - DNA, 18 bp, Artificial Sequence
  Oligonucleotide Primer
caggaaacagctatgacc

SEQ ID NO: 34 - DNA, 22 bp, Artificial Sequence
  Oligonucleotide Primer
tgacttatatactcgtgtcaac

SEQ ID NO: 35 - DNA, 21 bp, Artificial Sequence
  Oligonucleotide Primer
ctgctaatgtctactgtttgt